SEQUENCE LISTING (4 sequences)

SEQ ID NO: 1 - DNA, 1182 bp, herpes simplex virus 2
atggggcgtttgacctccggcgtcgggacggcggccctgctagttgtcgcggtgggactccgcgtcgtctgcgccaaatacgccttagcagacccctcgcttaagatggccgatcccaatcgatttcgcgggaagaaccttccggttttggaccagctgaccgacccccccggggtgaagcgtgtttaccacattcagccgagcctggaggacccgttccagccccccagcatcccgatcactgtgtactacgcagtgctggaacgtgcctgccgcagcgtgctcctacatgccccatcggaggccccccagatcgtgcgcggggcttcggacgaggcccgaaagcacacgtacaacctgaccatcgcctggtatcgcatgggagacaattgcgctatccccatcacggttatggaatacaccgagtgcccctacaacaagtcgttgggggtctgccccatccgaacgcagccccgctggagctactatgacagctttagcgccgtcagcgaggataacctgggattcctgatgcacgcccccgccttcgagaccgcgggtacgtacctgcggctagtgaagataaacgactggacggagatcacacaatttatcctggagcaccgggcccgcgcctcctgcaagtacgctctccccctgcgcatccccccggcagcgtgcctcacctcgaaggcctaccaacagggcgtgacggtcgacagcatcgggatgctaccccgctttatccccgaaaaccagcgcaccgtcgccctatacagcttaaaaatcgccgggtggcacggccccaagcccccgtacaccagcaccctgctgccgccggagctgtccgacaccaccaacgccacgcaacccgaactcgttccggaagaccccgaggactcggccctcttagaggatcccgccgggacggtgtcttcgcagatccccccaaactggcacatcccgtcgatccaggacgtcgcgccgcaccacgcccccgccgcccccagcaacccgggcctgatcatcggcgcgctggccggcagtaccctggcggtgctggtcatcggcggtattgcgttttgggtacgccgccgcgctcagatggcccccaagcgcctacgtctcccccacatccgggatgacgacgcgcccccctcgcaccagccattgttttactag

SEQ ID NO: 2 - protein, 393 aa, herpes simplex virus 2
MGRLTSGVGTAALLVVAVGLRVVCAKYALADPSLKMADPNRFRGKNLPVLDQLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEITQFILEHRARASCKYALPLRIPPAACLTSKAYQQGVTVDSIGMLPRFIPENQRTVALYSLKIAGWHGPKPPYTSTLLPPELSDTTNATQPELVPEDPEDSALLEDPAGTVSSQIPPNWHIPSIQDVAPHHAPAAPSNPGLIIGALAGSTLAVLVIGGIAFWVRRRAQMAPKRLRLPHIRDDDAPPSHQPLFY

SEQ ID NO: 3 - DNA, 1203 bp, Artificial Sequence
  recombinant
aagcttgccgccaccatgggacgtctgacgtcgggagtcggaacggctgctctgctggtcgtcgctgtgggactgcgcgtcgtctgcgctaaatacgctctggctgacccctcgctgaagatggctgatcccaatcgatttcgcggaaagaacctgcccgtcctggaccagctgacggacccccccggagtgaagcgtgtctaccacatccagccctcgctggaagacccctttcagcccccctcgatccccatcacggtgtactacgctgtgctggaacgtgcttgccgctcggtgctgctgcatgctccctcggaagctccccagatcgtgcgcggagcttcggacgaagctcgaaagcacacgtacaacctgacgatcgcttggtatcgcatgggagacaattgcgctatccccatcacggtcatggaatacacggaatgcccctacaacaagtcgctgggagtctgccccatccgaacgcagccccgctggtcgtactatgactcgttttcggctgtctcggaagataacctgggatttctgatgcacgctcccgcttttgaaacggctggaacgtacctgcgactggtgaagatcaacgactggacggaaatcacgcaatttatcctggaacaccgagctcgcgcttcgtgcaagtacgctctgcccctgcgcatcccccccgctgcttgcctgacgtcgaaggcttaccaacagggagtgacggtcgactcgatcggaatgctgccccgctttatccccgaaaaccagcgcacggtcgctctgtactcgctgaaaatcgctggatggcacggacccaagcccccctacacgtcgacgctgctgccccccgaactgtcggacacgacgaacgctacgcaacccgaactggtccccgaagaccccgaagactcggctctgctggaagatcccgctggaacggtgtcgtcgcagatcccccccaactggcacatcccctcgatccaggacgtcgctccccaccacgctcccgctgctccctcgaaccccggactgatcatcggagctctggctggatcgacgctggctgtgctggtcatcggaggaatcgctttttgggtccgccgccgcgctcagatggctcccaagcgcctgcgtctgccccacatccgagatgacgacgctcccccctcgcaccagcccctgttttactagctcgag

SEQ ID NO: 4 - DNA, 1173 bp, Artificial Sequence
  recombinant
aagcttgccgccaccatgcagatctttgtgaagacgctgacgggaaagacgatcacgctggaagtggaaccctcggacacgatcgaaaacgtgaaggctaagatccaggacaaggaaggaatcccccccgaccagcagagactgatctttgctggaaagcagctggaagacggacgcacgctgtcggactacaacatccagaaggaatcgacgctgcacctggtgctgagactgcgcggagctgctaaatacgctctggctgacccctcgcttaagatggctgatcccaatcgatttcgcggaaagaacctgcccgtcctggaccagctgacggacccccccggagtgaagcgtgtctaccacatccagccctcgctggaagacccctttcagcccccctcgatccccatcacggtgtactacgctgtgctggaacgtgcttgccgctcggtgctgctgcatgctccctcggaagctccccagatcgtgcgcggagcttcggacgaagctcgaaagcacacgtacaacctgacgatcgcttggtatcgcatgggagacaattgcgctatccccatcacggtcatggaatacacggaatgcccctacaacaagtcgctgggagtctgccccatccgaacgcagccccgctggtcgtactatgactcgttttcggctgtctcggaagataacctgggatttctgatgcacgctcccgcttttgaaacggctggaacgtacctgcgactggtgaagatcaacgactggacggaaatcacgcaatttatcctggaacaccgagctcgcgcttcgtgcaagtacgctctgcccctgcgcatcccccccgctgcttgcctgacgtcgaaggcttaccaacagggagtgacggtcgactcgatcggaatgctgccccgctttatccccgaaaaccagcgcacggtcgctctgtactcgctgaaaatcgctggatggcacggacccaagcccccctacacgtcgacgctgctgccccccgaactgtcggacacgacgaacgctacgcaacccgaactggtccccgaagaccccgaagactcggctctgctggaagatcccgctggaacggtgtcgtcgcagatcccccccaactggcacatcccctcgatccaggacgtcgctccccaccactagctcgag

(19) United States
(12) Patent Application Publication
(10) Pub. No.: US 2017/0224808 A1
(43) Pub. Date: Aug. 10, 2017

(54) THERAPEUTIC COMPOSITIOJNS AND METHODS FOR INDUCING AN IMMUNE RESPONSE TO HERPES SIMPLEX VIRUS TYPE 2 (HSV-2)

(71) Applicant: Admedus Vaccines Pty LTD, Woolloongabba (AU)

(72) Inventors: Julie Dutton, Toowoomba (AU); Ian Frazer, St. Lucia (AU)

(21) Appl. No.: 15/514,922

(22) PCT Filed: Oct. 1, 2015
(86) PCT No.: PCT/AU2015/050596

§ 371 Date: Mar. 28, 2017

(30) Foreign Application Priority Data
Oct. 1, 2014 (AU) 2014903921

(51) Int. Cl. A61K 39/245; C07K 14/005; C12N 7/00

(52) U.S. Cl. CPC A61K 39/245; C12N 7/00; C07K 14/005; C12N 2710/16022; C12N 2710/16034; C07K 2319/95; A61K 2039/545

(57) ABSTRACT

Disclosed are therapeutic compositions and methods for inducing an immune response to herpes simplex virus type 2 (HSV-2). More particularly, the invention relates to a method for inducing an immune response in a subject by introducing and expressing an HSV gD2-encoding DNA vaccine.

### FIELD OF THE INVENTION

This invention relates generally to the field of therapeutic compositions and methods for inducing an immune response to herpes simplex virus type 2 (HSV-2). More particularly, the invention relates to a method for inducing an immune response in a subject by introducing and expressing an HSV gD2-encoding DNA vaccine.

### BACKGROUND OF THE INVENTION

Herpes simplex virus 2 (HSV-2) is a member of the herpesvirus family, Herpesviridae, and is a major cause of genital ulcer diseases. The virus infects over 500 million people around the world (Looker et al, 2008). HSV-2 reaches a latent state in the sensory nerve root ganglia and reactivates when the immune function of the body declines, causing recurrent episodes (Gupta et al, 2007). However, the mechanisms that govern the viral latency remain elusive. Although genital herpes is a highly prevalent disease worldwide, no therapeutics against HSV-2 infection are currently available.

1.1 HSV-2 Pathogenesis

HSV-2 entry requires the complexation of viral glycoprotein D (gD2) with its receptors. The gD2 receptors include herpesvirus entry mediator (HVEM), nectin-1 and -2, as well as specific sites in heparin sulfate (Spear et al, 2000). During acute HSV infection gD2 interacts with HVEM which causes a decrease in the subsequent CD8⁺ recall response at the genital mucosa (Kopp et al, 2012).

HSV-2 also alters the innate immune responses by decreasing the level of type I interferon (i.e., IFN-α and IFN-β) and increasing the level of type II interferon (i.e., IFN-γ) (Peng et al, 2009). It is proposed that HSV-2 also blocks dendritic cell (DC) maturation and induces dendritic cell (DC) apoptosis and triggers the release of proinflammatory cytokines (Stefanidou et al, 2013; and Peretti et al, 2005). HSV-2 reactivation leads to recurrent episodes, ranging from mild to severe cases.

Symptoms of HSV infection include watery blisters in the skin or mucous membranes of the genitals. Lesions heal with a scab characteristic of herpetic disease.

1.2 Innate and Adaptive Immune Responses to HSV-2

A powerful and robust immune response to HSV-2 requires both the innate and the adaptive immune responses. The primary function of the adapative immune response is in viral clearance and generation of long-term memory, which has been the center of significant research attention. The interaction between the virus and innate immune cells (e.g., mononuclear phagocytes, dendritic cells (DC), and NKT cells) initiates the immune response via pattern recognition receptors (PRR). PRR recognize pathogen-associated molecular patterns (PAMP), for example, viral DNA and RNA. Toll-like receptors (TLR) are a major class of PRR and are expressed by innate immune cells, functioning to elicit an immune response.

The adaptive immune response consists of both cellular and humoral immunity. The main function of the adaptive immune response is to eliminate pathogens (e.g. viruses) and induce long-term memory against pathogenic antigens. Generally, the adaptive immune response is triggered by the innate immune response. Both CD4⁺ T cells and CD8⁺ T cells are required to elicit an effective HSV-2 specific immune response (see, Tilton et al., 2008).

Cytotoxic immunity complements the humoral system by eliminating cells infected with a pathogen (e.g., HSV-2 virus), and removing the intracellular pathogens, such as viruses. It has proven challenging to present an exogenously administered antigen in adequate concentrations, in conjunction with class I major histocompatibility complex (MHC) molecules to elicit an adequate immune response. This has severely hindered the development of vaccines against weakly immunogenic viral proteins (e.g., HSV-2).

In immunizing against agents, such as viruses, for which antibodies have been shown to enhance infectivity it would be desirable to provide a cellular immune response alone. Specifically, it is recognized that a cellular immune response to HSV-2 will be important for both the prevention of disease, and the control of recurrent disease (U.S. Pat. No. 8,828,408). It would also be useful to provide such a response against both chronic and latent viral infections.

1.3 Current Vaccine Formulations Against HSV-2

Several different vaccine formulation strategies have been considered for immunization against HSV infection, including inactivated vaccines, live attenuated vaccines, replication defective vaccines, subunit vaccines, peptide vaccines, live vector vaccines and DNA vaccines. However, no single strategy has yet proven successful.

The use of synthetic peptide vaccines has severe downfalls, at least because often peptides do not readily associate with MHC molecules, have a short serum half-life, are rapidly proteolyzed, and do not specifically localize to antigen-presenting monocytes and macrophages.

Inactivated virus vaccines are generally poorly immunogenic and have low efficacy. Further, such vaccines are reported to demonstrate potential to increase susceptibility of cancer and thus, are not currently being pursued.

Although live attenuated viruses have the ability to exert effective protection against HSV-2, clinical trials revealed that reoccurrence of the virus occurs in all but 37.5% of patients. HSV-2 ICPO⁻ mutant viruses reportedly induce a 10 to 100 times greater protection against genital herpes than the gD2 subunit vaccine (Halford et al, 2011), and thus show great promise against the disease. Another promising live attenuated HSV-2 vaccine is HSV-2 gD27, with point mutations at amino acids 215, 222 and 223. The variant polynucleotide is characterized by a loss-of-function in its ability to interact with the nectin-1 receptor. A significant disadvantage of live attenuated virus, however, is the ability of the virus to revert back to the wild-type phenotype.

Accordingly, there is a need for a method of eliciting a safe and effective immune response to an HSV-2 viral antigen. Moreover, there is a clear need for a method that will associate these antigens with class 1 MHC molecules on the cell surface of APC, to elicit a cytotoxic T cell response, avoid anaphylaxis and proteolysis of the material in the serum, and facilitate localization of the material to monocytes and macrophages (as discussed in U.S. Pat. No. 8,828,408).

1.4 Codon Optimization Based on Immune Response Preference

The present inventors previously disclosed in WO 2004/042059 a strategy for enhancing or reducing the quality of a selected phenotype that is displayed, or proposed to be displayed, by an organism of interest. The strategy involves codon modification of a polynucleotide that encodes a phenotype-associated polypeptide that either by itself, or in association with other molecules, in the organism of interest, imparts or confers the selected phenotype upon the organism. Unlike previous methods, however, this strategy does not rely on data that provide a ranking of synonymous codons according to their preference of usage in an organism or class of organism. Nor does it rely on data that provide a ranking of synonymous codons according to their translation efficiencies in one or more cells of the organism or class of organisms. Instead, it relies on ranking individual synonymous codons that code for an amino acid in the phenotype-associated polypeptide according to their preference of usage by the organism, or class of organisms, or by a part thereof for producing the selected phenotype.

The present inventors were then able to determine an immune response preference ranking of individual synonymous codons in mammals, as described in detail in WO 2009/049350. Comparison of the immune response preferences described in WO 2009/049350 with the translational efficiencies derived from codon usage frequency values for mammalian cells in general as determined by Seed (see U.S. Pat. Nos. 5,786,464 and 5,795,737) reveals several differences in the ranking of codons.

### SUMMARY OF THE INVENTION

The present invention is predicated in part on the surprising discovery that dermal administration of a binary nucleic acid construct system with enhanced production of qualitatively different forms of HSV gD2 elicits a significant delayed type hypersensitivity (DTH) response in a dose-dependent manner. Based on the unexpectedly strong cellular immune response elicited by this construct system, it is proposed that it would be particularly suited to therapeutic applications for combating HSV-2 infections, as described hereafter.

Accordingly, in one aspect, the present invention provides methods for treating a herpes simplex virus-2 (HSV-2) infection in a subject. These methods generally comprise administering concurrently to the subject an effective amount of a construct system that comprises a first construct and a second construct, wherein the first construct comprises a first synthetic coding sequence that is distinguished from a wild-type HSV gD2 coding sequence by replacement of selected codons in the wild-type HSV gD2 coding sequence with synonymous codons that have a higher immune response preference than the selected codons, wherein codon replacements are selected from Table 1 and wherein at least 70% of the codons of the first synthetic coding sequence are synonymous codons according to Table 1, and wherein the first synthetic coding sequence is operably connected to a regulatory nucleic acid sequence, and wherein the second construct comprises a second synthetic coding sequence that is distinguished from a wild-type HSV gD2 coding sequence by replacement of selected codons in the wild-type HSV gD2 coding sequence with synonymous codons that have a higher immune response preference than the selected codons and wherein codon replacements are selected from Table 1 and wherein at least 70% of the codons of the second synthetic coding sequence are synonymous codons according to Table 1, and wherein the second synthetic coding sequence is operably connected to a regulatory nucleic acid sequence and to a nucleic acid sequence that encodes a protein-destabilizing element that increases processing and presentation of the polypeptide through the class I major histocompatibility (MHC) pathway, wherein TABLE 1 is as follows:

TABLE 1

| First | Synonymous | First | Synonymous | First | Synonymous |
|---|---|---|---|---|---|
| Codon | Codon | Codon | Codon | Codon | Codon |
| Ala^{GCG} | Ala^{GCT} | Ile^{ATA} | Ile^{ATC} | Ser^{AGT} | Ser^{TCG} |
| Ala^{GCG} | Ala^{GCC} | Ile^{ATA} | Ile^{ATT} | Ser^{AGT} | Ser^{TCT} |
| Ala^{GCA} | Ala^{GCT} | Ile^{ATT} | Ile^{ATC} | Ser^{AGT} | Ser^{TCA} |
| Ala^{GCA} | Ala^{GCC} | | | Ser^{AGT} | Ser^{TCC} |
| Ala^{GCC} | Ala^{GCT} | Leu^{TTA} | Leu^{CTG} | Ser^{AGC} | Ser^{TCG} |
| | | Leu^{TTA} | Leu^{CTC} | Ser^{AGC} | Ser^{TCT} |
| Arg^{CGG} | Arg^{CGA} | Leu^{TTA} | Leu^{CTA} | Ser^{AGC} | Ser^{TCA} |
| Arg^{CGG} | Arg^{CGC} | Leu^{TTA} | Leu^{CTT} | Ser^{AGC} | Ser^{TCC} |
| Arg^{CGG} | Arg^{CGT} | Leu^{TTA} | Leu^{TTG} | Ser^{TCC} | Ser^{TCG} |
| Arg^{CGG} | Arg^{AGA} | Leu^{TTG} | Leu^{CTG} | Ser^{TCA} | Ser^{TCG} |
| Arg^{AGG} | Arg^{CGA} | Leu^{TTG} | Leu^{CTC} | Ser^{TCT} | Ser^{TCG} |
| Arg^{AGG} | Arg^{CGC} | Leu^{TTG} | Leu^{CTA} | | |
| Arg^{AGG} | Arg^{CGT} | Leu^{TTG} | Leu^{CTT} | Thr^{ACT} | Thr^{ACG} |
| Arg^{AGG} | Arg^{AGA} | Leu^{CTT} | Leu^{CTG} | Thr^{ACT} | Thr^{ACC} |
| | | Leu^{CTT} | Leu^{CTC} | Thr^{ACT} | Thr^{ACA} |
| Asn^{AAT} | Asn^{AAC} | Leu^{CTA} | Leu^{CTG} | Thr^{ACA} | Thr^{ACG} |
| | | Leu^{CTA} | Leu^{CTC} | Thr^{ACA} | Thr^{ACC} |
| Asp^{GAT} | Asp^{GAC} | | | Thr^{ACC} | Thr^{ACG} |
| | | Phe^{TTC} | Phe^{TTT} | | |
| Cys^{TGT} | Cys^{TGC} | | | Tyr^{TAT} | Tyr^{TAC} |
| | | Pro^{CCG} | Pro^{CCC} | | |
| Glu^{GAG} | Glu^{GAA} | Pro^{CCG} | Pro^{CCT} | Val^{GTA} | Val^{GTG} |
| | | Pro^{CCA} | Pro^{CCC} | Val^{GTA} | Val^{GTC} |
| Gly^{GGC} | Gly^{GGA} | Pro^{CCA} | Pro^{CCT} | Val^{GTA} | Val^{GTT} |
| Gly^{GGT} | Gly^{GGA} | Pro^{CCT} | Pro^{CCC} | Val^{GTT} | Val^{GTG} |
| Gly^{GGG} | Gly^{GGA} | | | Val^{GTT} | Val^{GTC} |

In some embodiments, the methods further comprise identifying that the subject has an HSV-2 infection prior to administering concurrently the first and second constructs.

In some embodiments, the protein-destabilizing element is selected from the group consisting of a destabilizing amino acid at the amino-terminus of the polypeptide, a PEST sequence and a ubiquitin molecule. Suitably, the protein-destabilizing element is a ubiquitin molecule.

Thus, by replacing codons of the wild-type HSV gD2 coding sequence with those identified in Table 1, an immune response (suitably a cellular immune response, which includes a DTH response) that is stronger or enhanced by at least about 110%, 150%, 200%, 300%, 400%, 500%, 600%, 700%, 800%, 900%, 1000% and all integer percentages in between, than that produced by the wild-type coding sequence under identical conditions is achievable. It is preferable, but not necessary, to replace all the codons of the wild-type HSV gD2 coding sequence with synonymous codons selected from Table 1. In some embodiments, the first synthetic coding sequence and the second synthetic coding sequence are each distinguished from the wild-type HSV gD2 coding sequence by the replacement of a number of selected codons with synonymous codons that have a higher immune response preference than the selected codons, so that at least 75%, 80%, 85%, 90%, 95%, 96%, 97%, 98%, 99% and all integer percentages in between, of the codons in the first synthetic coding sequence and the second synthetic coding sequence are synonymous codons selected from Table 1. In some embodiments, the first and second synthetic coding sequence comprise or consist of the same nucleic acid sequence. In other embodiments, the first and second synthetic coding sequences comprise or consist of different nucleic acid sequences. In illustrative examples of this type, the first synthetic coding sequence comprises different codon replacements relative to the second synthetic coding sequence. In illustrative examples, the first synthetic coding sequence comprises a different number of codon replacements relative to the second synthetic coding sequence.

In some embodiments, the first and second synthetic coding sequence correspond to full length HSV gD2 coding sequence. In other embodiments, the first synthetic coding sequence corresponds to full length HSV gD2 coding sequence, and the second synthetic coding sequence corresponds to a portion of the HSV gD2 coding sequence. In still other embodiments, the first synthetic coding sequence corresponds to a portion of the HSV gD2 coding sequence, and the second synthetic coding sequence corresponds to full length HSV gD2 coding sequence. Yet in other embodiments the first and second synthetic coding sequence each corresponds to at least a portion of the HSV gD2 coding sequence. Suitably, the portion of HSV gD2 coding sequence encodes amino acid residues 25-331 of the full length HSV gD2 polypeptide. In specific embodiments, the first synthetic coding sequence corresponds to the full length HSV gD2 coding sequence, and the second synthetic coding sequence corresponds to a portion of the HSV gD2 coding sequence encoding amino acid residues 25-331 of the full length HSV gD2 polypeptide.

In specific examples, the first synthetic coding sequence comprises the sequence set forth in SEQ ID NO: 3, and the second synthetic coding sequence comprises the sequence set forth in SEQ ID NO: 4.

The first construct and the second construct may be contained in the same vector or in a separate vector. In some embodiments the vectors are free of any non-essential sequences (e.g., a signal or targeting sequence).

In some embodiments, the first construct and the second construct are contained in a pharmaceutical composition that optionally comprises a pharmaceutically acceptable excipient and/or carrier. Accordingly, in another aspect, the invention provides immunogenic pharmaceutical compositions that are useful for treating an HSV-2 infection. In some embodiments of this aspect, the compositions are formulated for dermal or subdermal administration (e.g., intradermal administration, transdermal administration, or subcutaneous administration). In specific embodiments, the compositions are formulated for intradermal administration. In some embodiments the dose of the construct system administered to a subject is at least about 30 μg per injection. In specific embodiments, doses of 30 μg, 50 μg, 100 μg, 150 μg, 200 μg, 250 μg, 300 μg, 500 μg, 750 μg, 1000 μg or more are suitable per injection. Suitably, the subject is subjected to several rounds of treatment. By way of example, the subject may receive 3 separate doses at fortnightly intervals. However, other treatment regimes are suitable and can be tailored to the needs of the subject.

In some embodiments, the composition is formulated with an adjuvant. In other embodiments the composition is formulated without the addition of any adjuvant.

In preferred embodiments, the subject is a human.

In another aspect, the present invention provides a use of a construct system as broadly defined above and elsewhere herein for treating an HSV-2 infection. In some embodiments, the construct system is prepared or manufactured as a medicament for this purpose.

### BRIEF DESCRIPTION OF THE DRAWINGS

FIG. 1 shows schematic maps of NTC8485-O2-gD2 and NTC8485-O2-Ubi-gD2tr. NTC8485 vector map showing the location of the first synthetic coding sequence (A) O2-gD2, and (B) O2-Ubi-gD2tr.

FIG. 2 shows photographs of the injection site of a subject after administration of 500 μg dose of COR-1 vaccine. Photographs were taken of the right arm injection site (A) immediately; (B) 45 minutes post injection; (C) 24 hours post injection; and (D) 48 hours post injection.

FIG. 3 shows photographs of the injection site of a subject after administration of 500 μg dose of COR-1 vaccine. Photographs were taken of the left arm injection site (A) immediately; (B) 45 minutes post injection; (C) 24 hours post injection; and (D) 48 hours post injection.

FIG. 4 shows photographs of the injection site of a subject after administration of 30 μg dose of COR-1 vaccine. Photographs were taken (A) immediately; (B) 45 minutes post injection; (C) 24 hours post injection; and (D) 48 hours post injection.

FIG. 5 shows photographs of the injection site of a subject after administration of 100 μg dose of COR-1 vaccine. Photographs were taken (A) immediately; (B) 45 minutes post injection; (C) 24 hours post injection; and (D) 48 hours post injection.

FIG. 6 shows photographs of the injection site of a subject after administration of 300 μg dose of COR-1 vaccine. Photographs were taken (A) immediately; (B) 45 minutes post injection; (C) 24 hours post injection; and (D) 48 hours post injection.

TABLE A
BRIEF DESCRIPTION OF THE SEQUENCES

| SEQUENCE ID | | |
|---|---|---|
| NUMBER | SEQUENCE | LENGTH |
| SEQ ID NO: 1 | HSV gD2 wild-type | 1182 nts |
| SEQ ID NO: 2 | HSV gD2 amino acid | 393 aa |
| SEQ ID NO: 3 | NTC8485-O2-gD2 | 1203 nts |
| SEQ ID NO: 4 | NTC8485-O2-Ubi-gD2tr | 1173 nts |

### DETAILED DESCRIPTION OF THE INVENTION

### I. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

By “about” is meant a quantity, level, value, frequency, percentage, dimension, size, or amount that varies by no more than 15%, and preferably by no more than 10%, 9%, 8%, 7%, 6%, 5%, 4%, 3%, 2%, 1% to a reference quantity, level, value, frequency, percentage, dimension, size, or amount.

The terms “administration concurrently” or “administering concurrently” or “co-administering” and the like refer to the administration of a single composition containing two or more actives, or the administration of each active as separate compositions and/or delivered by separate routes either contemporaneously or simultaneously or sequentially within a short enough period of time that the effective result is equivalent to that obtained when all such actives are administered as a single composition. By “simultaneously” is meant that the active agents are administered at substantially the same time, and desirably together in the same formulation. By “contemporaneously” it is meant that the active agents are administered closely in time, e.g., one agent is administered within from about one minute to within about one day before or after another. Any contemporaneous time is useful. However, it will often be the case that when not administered simultaneously, the agents will be administered within about one minute to within about eight hours and preferably within less than about one to about four hours. When administered contemporaneously, the agents are suitably administered at the same site on the subject. The term “same site” includes the exact location, but can be within about 0.5 to about 15 centimeters, preferably from within about 0.5 to about 5 centimeters. The term “separately” as used herein means that the agents are administered at an interval, for example at an interval of about a day to several weeks or months. The active agents may be administered in either order. The term “sequentially” as used herein means that the agents are administered in sequence, for example at an interval or intervals of minutes, hours, days or weeks. If appropriate the active agents may be administered in a regular repeating cycle.

As used herein, “and/or” refers to and encompasses any and all possible combinations of one or more of the associated listed items, as well as the lack of combinations when interpreted in the alternative (or).

The terms “antigen” and “epitope” are well understood in the art and refer to the portion of a macromolecule which is specifically recognized by a component of the immune system, e.g., an antibody or a T-cell antigen receptor. Epitopes are recognized by antibodies in solution, e.g., free from other molecules. Epitopes are recognized by T-cell antigen receptor when the epitope is associated with a class I or class II major histocompatability complex molecule. A “CTL epitope” is an epitope recognized by a cytotoxic T lymphocyte (usually a CD8⁺ cell) when the epitope is presented on a cell surface in association with an MHC Class I molecule.

It will be understood that the term “between” when used in reference to a range of numerical values encompasses the numerical values at each endpoint of the range. For example, a composition comprising between 30 μg and about 1000 μg of synthetic construct is inclusive of a composition comprising 30 μg of synthetic construct and a composition comprising 1000 μg of synthetic construct.

As used herein, the term “cis-acting sequence” or “cis-regulatory region” or similar term shall be taken to mean any sequence of nucleotides which is derived from an expressible genetic sequence wherein the expression of the genetic sequence is regulated, at least in part, by the sequence of nucleotides. Those skilled in the art will be aware that a cis-regulatory region may be capable of activating, silencing, enhancing, repressing or otherwise altering the level of expression and/or cell-type-specificity and/or developmental specificity of any structural gene sequence.

Throughout this specification, unless the context requires otherwise, the words “comprise,” “comprises” and “comprising” will be understood to imply the inclusion of a stated step or element or group of steps or elements but not the exclusion of any other step or element or group of steps or elements.

By “coding sequence” is meant any nucleic acid sequence that contributes to the code for the polypeptide product of a gene. By contrast, the term “non-coding sequence” refers to any nucleic acid sequence that does not contribute to the code for the polypeptide product of a gene.

The term “delayed type hypersensitivity” (also termed type IV hypersensitivity) as used herein refers to a cell-mediated immune response comprising CD4⁺ and/or CD8⁺ T cells. CD4⁺ helper T cells recognize antigens presented by Class II MHC molecules on antigen-presenting cells (APC). The APC in this case are often IL-12-secreting macrophages, which stimulate the proliferation of further CD4⁺ Th1 cells. These CD4⁺ T cells, in turn, secrete IL-2 and IFN-γ, further inducing the release of other Th1 cytokines, and thus mediating a substantial cellular immune response. The CD8⁺ T cells function to destroy target cells on contact, whereas activated macrophages produce hydrolytic enzymes on exposure to intracellular pathogens. DTH responses in the skin are commonly used to assess cellular immunity in vivo (see, Pichler et al, 2011). Specifically, after dermal or subdermal administration, suitably intradermal administration, of an antigen, occurrence of induration and erythema at about 48 hours post-injection are strongly indicative of a positive DTH reaction, and a substantial cellular immune response.

By “effective amount,” in the context of modulating an immune response or treating or preventing a disease or condition, is meant the administration of that amount of composition to an individual in need thereof, either in a single dose or as part of a series, that is effective for achieving that modulation, treatment or prevention. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

It will be understood that “eliciting” or “inducing” an immune response as contemplated herein includes stimulating a new immune response and/or enhancing a previously existing immune response.

As used herein, the terms “encode,” “encoding” and the like refer to the capacity of a nucleic acid to provide for another nucleic acid or a polypeptide. For example, a nucleic acid sequence is said to “encode” a polypeptide if it can be transcribed and/or translated to produce the polypeptide or if it can be processed into a form that can be transcribed and/or translated to produce the polypeptide. Such a nucleic acid sequence may include a coding sequence or both a coding sequence and a non-coding sequence. Thus, the terms “encode,” “encoding” and the like include an RNA product resulting from transcription of a DNA molecule, a protein resulting from translation of an RNA molecule, a protein resulting from transcription of a DNA molecule to form an RNA product and the subsequent translation of the RNA product, or a protein resulting from transcription of a DNA molecule to provide an RNA product, processing of the RNA product to provide a processed RNA product (e.g., mRNA) and the subsequent translation of the processed RNA product.

The terms “enhancing an immune response,” “producing a stronger immune response” and the like refer to increasing an animal's capacity to respond to an HSV gD2 polypeptide, which can be determined for example by detecting an increase in the number, activity, and ability of the animal's cells that are primed to attack such an antigen and/or an increase in the titer or activity of antibodies in the animal, which are immuno-interactive with the HSV gD2 polypeptide. Strength of immune response can be measured by standard immunoassays including: direct measurement of antibody titers or peripheral blood lymphocytes; cytolytic T lymphocyte assays; assays of natural killer cell cytotoxicity; cell proliferation assays including lymphoproliferation (lymphocyte activation) assays; immunoassays of immune cell subsets; assays of T-lymphocytes specific for the antigen in a sensitized subject; skin tests for cell-mediated immunity; etc. Such assays are well known in the art. See, e.g., Erickson et al., 1993, J. Immunol. 151:4189-4199; Doe et al., 1994, Eur. J. Immunol. 24:2369-2376. Recent methods of measuring cell-mediated immune response include measurement of intracellular cytokines or cytokine secretion by T-cell populations, or by measurement of epitope specific T-cells (e.g., by the tetramer technique) (reviewed by McMichael, A. J., and O'Callaghan, C. A., 1998, J. Exp. Med. 187(9)1367-1371; Mcheyzer-Williams, M. G., et al., 1996, Immunol. Rev. 150:5-21; Lalvani, A., et al., 1997, J. Exp. Med. 186:859-865). Any statistically significant increase in strength of immune response as measured for example by immunoassay is considered an “enhanced immune response” or “immunoenhancement” as used herein. Enhanced immune response is also indicated by physical manifestations such as inflammation, as well as healing of systemic and local infections, and reduction of symptoms in disease, i.e., herpetic and warts. Such physical manifestations also encompass “enhanced immune response” or “immunoenhancement” as used herein.

The term “expression” with respect to a gene sequence refers to transcription of the gene and, as appropriate, translation of the resulting mRNA transcript to a protein. Thus, as will be clear from the context, expression of a coding sequence results from transcription and translation of the coding sequence. Conversely, expression of a non-coding sequence results from the transcription of the non-coding sequence.

By “expression vector” is meant any autonomous genetic element capable of directing the synthesis of a protein encoded by the vector. Such expression vectors are known by practitioners in the art.

The term “gene” as used herein refers to any and all discrete coding regions of a genome, as well as associated non-coding and regulatory regions. The gene is also intended to mean an open reading frame encoding one or more specific polypeptides, and optionally comprising one or more introns, and adjacent 5′ and 3′ non-coding nucleotide sequences involved in the regulation of expression. In this regard, the gene may further comprise regulatory nucleic acids such as promoters, enhancers, termination and/or polyadenylation signals that are naturally associated with a given gene, or heterologous control signals. Genes may or may not be capable of being used to produce a functional protein. Genes can include both coding and non-coding regions.

As used herein, the term “HSV gD2” (or “herpes simplex virus type-2 glycoprotein D”) in the context of a nucleic acid or amino acid sequence, refers to a full or partial length HSV gD2 coding sequence or a full or partial length HSV gD2 amino acid sequence (e.g., a full or partial length gD2 gene of HSV strain HG52, genome strain NC_001798, a protein expression product thereof). In some embodiments, a synthetic coding sequence encodes at least about 5, 6, 7, 8, 9, 10, 11, 12, 13, 14, 15, 16, 17, 18, 19, 20, 21, 22, 23, 24, 25, 30, 40, 50, 60, 70, 80, 90, 100, 120, 150, 200, 250, 300 or 350 contiguous amino acid residues, or almost up to the total number of amino acids present in a full-length HSV gD2 amino acid sequence (393 amino acid residues). In some embodiments, the synthetic coding sequence encodes a plurality of portions of the HSV gD2 polypeptide, wherein the portions are the same or different. In illustrative examples of this type, the synthetic coding sequence encodes a multi-epitope fusion protein. A number of factors can influence the choice of portion size. For example, the size of individual portions encoded by the synthetic coding sequence can be chosen such that it includes, or corresponds to the size of, T cell epitopes and/or B cell epitopes, and their processing requirements. Practitioners in the art will recognize that class I-restricted T cell epitopes are typically between 8 and 10 amino acid residues in length and if placed next to unnatural flanking residues, such epitopes can generally require 2 to 3 natural flanking amino acid residues to ensure that they are efficiently processed and presented. Class II-restricted T cell epitopes usually range between 12 and 25 amino acid residues in length and may not require natural flanking residues for efficient proteolytic processing although it is believed that natural flanking residues may play a role. Another important feature of class II-restricted epitopes is that they generally contain a core of 9-10 amino acid residues in the middle which bind specifically to class II MHC molecules with flanking sequences either side of this core stabilizing binding by associating with conserved structures on either side of class II MHC antigens in a sequence independent manner. Thus the functional region of class II-restricted epitopes is typically less than about 15 amino acid residues long. The size of linear B cell epitopes and the factors effecting their processing, like class II-restricted epitopes, are quite variable although such epitopes are frequently smaller in size than 15 amino acid residues. From the foregoing, it is advantageous, but not essential, that the size of individual portions of the HSV gD2 polypeptide is at least 6, 7, 8, 9, 10, 11, 12, 13, 14, 15, 20, 25, 30 amino acid residues. Suitably, the size of individual portions is no more than about 500, 200, 100, 80, 60, 50, 40 amino acid residues. In certain advantageous embodiments, the size of individual portions is sufficient for presentation by an antigen-presenting cell of a T cell and/or a B cell epitope contained within the peptide.

“Immune response” or “immunological response” refers to the concerted action of any one or more of lymphocytes, antigen-presenting cells, phagocytic cells, granulocytes, and soluble macromolecules produced by the above cells or the liver (including antibodies, cytokines, and complement) that results in selective damage to, destruction of, or elimination from the body of invading pathogens, cells or tissues infected with pathogens. In some embodiments, an “immune response’ encompasses the development in an individual of a humoral and/or a cellular immune response to a polypeptide that is encoded by an introduced synthetic coding sequence of the invention. As known in the art, the terms “humoral immune response” includes and encompasses an immune response mediated by antibody molecules, while a “cellular immune response” includes and encompasses an immune response mediated by T-lymphocytes and/or other white blood cells. Hence, an immunological response may include one or more of the following effects: the production of antibodies by B-cells; and/or the activation of suppressor T-cells and/or memory/effector T-cells directed specifically to an antigen or antigens present in the composition or vaccine of interest. In some embodiments, these responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art. (See, e.g., Montefiori et al., 1988, J Clin Microbiol. 26:231-235; Dreyer et al., 1999, AIDS Res Hum Retroviruses 15(17):1563-1571). The innate immune system of mammals also recognizes and responds to molecular features of pathogenic organisms and cancer cells via activation of Toll-like receptors and similar receptor molecules on immune cells. Upon activation of the innate immune system, various non-adaptive immune response cells are activated to, e.g., produce various cytokines, lymphokines and chemokines. Cells activated by an innate immune response include immature and mature dendritic cells of, for example, the monocyte and plasmacytoid lineage (MDC, PDC), as well as gamma, delta, alpha and beta T cells and B cells and the like. Thus, the present invention also contemplates an immune response wherein the immune response involves both an innate and adaptive response.

A composition is “immunogenic” if it is capable of either: a) generating an immune response against an HSV gD2 polypeptide in an individual; or b) reconstituting, boosting, or maintaining an immune response in an individual beyond what would occur if the agent or composition was not administered. An agent or composition is immunogenic if it is capable of attaining either of these criteria when administered in single or multiple doses. The immune response may include a cellular immune response and/or humoral immune response in a subject.

Throughout this specification, unless the context requires otherwise, the words “include,” “includes” and “including” will be understood to imply the inclusion of a stated step or element or group of steps or elements but not the exclusion of any other step or element or group of steps or elements.

As used herein, the term “mammal” refers to any mammal including, without limitation, humans and other primates, including non-human primates such as chimpanzees and other apes and monkey species; farm animals such as cattle, sheep, pigs, goats and horses; domestic mammals such as dogs and cats; and laboratory animals including rodents such as mice, rats and guinea pigs. The term does not denote a particular age. Thus, both adult and newborn individuals are intended to be covered.

The terms “operably connected,” “operably linked” and the like as used herein refer to an arrangement of elements wherein the components so described are configured so as to perform their usual function. Thus, a given regulatory nucleic acid such as a promoter operably linked to a coding sequence is capable of effecting the expression of the coding sequence when the proper enzymes are present. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence. Terms such as “operably connected,” therefore, include placing a structural gene under the regulatory control of a promoter, which then controls the transcription and optionally translation of the gene. In the construction of heterologous promoter/structural gene combinations, it is generally preferred to position the genetic sequence or promoter at a distance from the gene transcription start site that is approximately the same as the distance between that genetic sequence or promoter and the gene it controls in its natural setting; i.e. the gene from which the genetic sequence or promoter is derived. As is known in the art, some variation in this distance can be accommodated without loss of function. Similarly, the preferred positioning of a promoter with respect to a heterologous gene to be placed under its control is defined by the positioning of the promoter in its natural setting; i.e., the genes from which it is derived. Alternatively, “operably connecting” a gD2 coding sequence to a nucleic acid sequence that encodes a protein-destabilizing element (PDE) encompasses positioning and/or orientation of the gD2 coding sequence relative to the PDE-encoding nucleic acid sequence so that (1) the coding sequence and the PDE-encoding nucleic acid sequence are transcribed together to form a single chimeric transcript and (2) the gD2 coding sequence is ‘in-frame’ with the PDE-encoding nucleic acid sequence to produce a chimeric open reading frame comprising the gD2 coding sequence and the PDE-encoding nucleic acid sequence.

The terms “open reading frame” and “ORF” refer to the amino acid sequence encoded between translation initiation and termination codons of a coding sequence. The terms “initiation codon” and “termination codon” refer to a unit of three adjacent nucleotides (‘codon’) in a coding sequence that specifies initiation and chain termination, respectively, of protein synthesis (mRNA translation).

By “pharmaceutically-acceptable carrier” is meant a solid or liquid filler, diluent or encapsulating substance that may be safely used in topical or systemic administration.

The term “polynucleotide” or “nucleic acid” as used herein designates mRNA, RNA, cRNA, cDNA or DNA. The term typically refers to oligonucleotides greater than 30 nucleotides in length.

“Polypeptide,” “peptide” and “protein” are used interchangeably herein to refer to a polymer of amino acid residues and to variants and synthetic analogues of the same. As used herein, the terms “polypeptide,” “peptide” and “protein” are not limited to a minimum length of the product. Thus, peptides, oligopeptides, dimers, multimers, and the like, are included within the definition. Both full-length proteins and fragments thereof are encompassed by the definition. The terms also include post expression modifications of a polypeptide, for example, glycosylation, acetylation, phosphorylation and the like. In some embodiments, a “polypeptide” refers to a protein which includes modifications, such as deletions, additions and substitutions (generally conservative in nature), to the native sequence, so long as the protein maintains the desired activity. These modifications may be deliberate, as through site-directed mutagenesis, or may be accidental, such as through mutations of hosts which produce the proteins or errors due to PCR amplification.

The terms “polypeptide variant,” and “variant” refer to polypeptides that vary from a reference polypeptide by the addition, deletion or substitution (generally conservative in nature) of at least one amino acid residue. Typically, variants retain a desired activity of the reference polypeptide, such as antigenic activity in inducing an immune response against an HSV gD2 polypeptide. In general, variant polypeptides are “substantially similar” or substantially identical” to the reference polypeptide, e.g., amino acid sequence identity or similarity of more than 50%, generally more than 60%-70%, even more particularly 80%-85% or more, such as at least 90%-95% or more, when the two sequences are aligned. Often, the variants will include the same number of amino acids but will include substitutions, as explained herein.

Reference herein to a “promoter” is to be taken in its broadest context and includes the transcriptional regulatory sequences of a classical genomic gene, including the TATA box which is required for accurate transcription initiation, with or without a CCAAT box sequence and additional regulatory elements (i.e. upstream activating sequences, enhancers and silencers) which alter gene expression in response to developmental and/or environmental stimuli, or in a tissue-specific or cell-type-specific manner. A promoter is usually, but not necessarily, positioned upstream or 5′, of a structural gene, the expression of which it regulates. Furthermore, the regulatory elements comprising a promoter are usually positioned within 2 kb of the start site of transcription of the gene. Preferred promoters according to the invention may contain additional copies of one or more specific regulatory elements to further enhance expression in a cell, and/or to alter the timing of expression of a structural gene to which it is operably connected.

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Be, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for Windows; available from Hitachi Software engineering Co., Ltd., South San Francisco, Calif., USA) using standard defaults as used in the reference manual accompanying the software.

“Similarity” refers to the percentage number of amino acids that are identical or constitute conservative substitutions as defined in Table 10. Similarity may be determined using sequence comparison programs such as GAP (Deveraux et al. 1984, Nucleic Acids Research 12, 387-395). In this way, sequences of a similar or substantially different length to those cited herein might be compared by insertion of gaps into the alignment, such gaps being determined, for example, by the comparison algorithm used by GAP.

Terms used to describe sequence relationships between two or more polynucleotides or polypeptides include “reference sequence”, “comparison window”, “sequence identity”, “percentage of sequence identity” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., “Current Protocols in Molecular Biology”, John Wiley & Sons Inc, 1994-1998, Chapter 15.

The term “synthetic coding sequence” as used herein refers to a polynucleotide that is formed by recombinant or synthetic techniques and typically includes polynucleotides that are not normally found in nature.

The term “synonymous codon” as used herein refers to a codon having a different nucleotide sequence than another codon but encoding the same amino acid as that other codon.

By “treatment,” “treat,” “treated” and the like is meant to include both therapeutic and prophylactic treatment.

By “vector” is meant a nucleic acid molecule, preferably a DNA molecule derived, for example, from a plasmid, bacteriophage, or plant virus, into which a nucleic acid sequence may be inserted or cloned. A vector preferably contains one or more unique restriction sites and may be capable of autonomous replication in a defined host cell including a target cell or tissue or a progenitor cell or tissue thereof, or be integrable with the genome of the defined host such that the cloned sequence is reproducible. Accordingly, the vector may be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. A vector system may comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. The vector may also include a selection marker such as an antibiotic resistance gene that can be used for selection of suitable transformants. Examples of such resistance genes are well known to those of skill in the art.

The terms “wild-type,” “natural,” “native” and the like with respect to an organism, polypeptide, or nucleic acid sequence, refer to an organism, polypeptide or nucleic acid sequence that is naturally occurring or available in at least one naturally occurring organism which is not changed, mutated, or otherwise manipulated by man.

### 2. Abbreviations

The following abbreviations are used throughout the application:

nt=nucleotide

nts=nucleotides

bp=base pair

aa=amino acid(s)

### 3. HSV gD2 Coding Sequences

The first and second synthetic coding sequences contemplated for use in the present invention encode proteinaceous molecules, representative examples of which include polypeptides and peptides. Wild-type HSV gD2 polypeptides are suitable for use in the present invention, although variant HSV gD2 polypeptides are also contemplated. In accordance with the present invention, the HSV gD2 polypeptides produced from the nucleic acid constructs of the invention are encoded by codon-optimized HSV gD2 coding sequences.

In some embodiments, a synthetic coding sequence is produced based on codon optimizing at least a portion of a wild-type HSV gD2 coding sequence, an illustrative example of which includes the HSV gD2 coding sequence of strain HG52 (genome strain NC_001798) which has the following nucleotide sequence:

[SEQ ID NO: 1]
ATGGGGCGTTTGACCTCCGGCGTCGGGACGGCGGCCCTGCTAGTTGTCGC
GGTGGGACTCCGCGTCGTCTGCGCCAAATACGCCTTAGCAGACCCCTCGC
TTAAGATGGCCGATCCCAATCGATTTCGCGGGAAGAACCTTCCGGTTTTG
GACCAGCTGACCGACCCCCCCGGGGTGAAGCGTGTTTACCACATTCAGCC
GAGCCTGGAGGACCCGTTCCAGCCCCCCAGCATCCCGATCACTGTGTACT
ACGCAGTGCTGGAACGTGCCTGCCGCAGCGTGCTCCTACATGCCCCATCG
GAGGCCCCCCAGATCGTGCGCGGGGCTTCGGACGAGGCCCGAAAGCACAC
GTACAACCTGACCATCGCCTGGTATCGCATGGGAGACAATTGCGCTATCC
CCATCACGGTTATGGAATACACCGAGTGCCCCTACAACAAGTCGTTGGGG
GTCTGCCCCATCCGAACGCAGCCCCGCTGGAGCTACTATGACAGCTTTAG
CGCCGTCAGCGAGGATAACCTGGGATTCCTGATGCACGCCCCCGCCTTCG
AGACCGCGGGTACGTACCTGCGGCTAGTGAAGATAAACGACTGGACGGAG
ATCACACAATTTATCCTGGAGCACCGGGCCCGCGCCTCCTGCAAGTACGC
TCTCCCCCTGCGCATCCCCCCGGCAGCGTGCCTCACCTCGAAGGCCTACC
AACAGGGCGTGACGGTCGACAGCATCGGGATGCTACCCCGCTTTATCCCC
GAAAACCAGCGCACCGTCGCCCTATACAGCTTAAAAATCGCCGGGTGGCA
CGGCCCCAAGCCCCCGTACACCAGCACCCTGCTGCCGCCGGAGCTGTCCG
ACACCACCAACGCCACGCAACCCGAACTCGTTCCGGAAGACCCCGAGGAC
TCGGCCCTCTTAGAGGATCCCGCCGGGACGGTGTCTTCGCAGATCCCCCC
AAACTGGCACATCCCGTCGATCCAGGACGTCGCGCCGCACCACGCCCCCG
CCGCCCCCAGCAACCCGGGCCTGATCATCGGCGCGCTGGCCGGCAGTACC
CTGGCGGTGCTGGTCATCGGCGGTATTGCGTTTTGGGTACGCCGCCGCGC
TCAGATGGCCCCCAAGCGCCTACGTCTCCCCCACATCCGGGATGACGACG
CGCCCCCCTCGCACCAGCCATTGTTTTACTAG.

This polynucleotide sequence set forth in SEQ ID NO: 1 encodes the following amino acid sequence (UniProt Accession No. NP044536):

[SEQ ID NO: 2]
MGRLTSGVGTAALLVVAVGLRVVCAKYALADPSLKMADPNRFRGKNLPVL
DQLTDPPGVKRVYHIQPSLEDPFQPPSIPITVYYAVLERACRSVLLHAPS
EAPQIVRGASDEARKHTYNLTIAWYRMGDNCAIPITVMEYTECPYNKSLG
VCPIRTQPRWSYYDSFSAVSEDNLGFLMHAPAFETAGTYLRLVKINDWTE
ITQFILEHRARASCKYALPLRIPPAACLTSKAYQQGVTVDSIGMLPRFIP
ENQRTVALYSLKIAGWHGPKPPYTSTLLPPELSDTTNATQPELVPEDPED
SALLEDPAGTVSSQIPPNWHIPSIQDVAPHHAPAAPSNPGLIIGALAGST
LAVLVIGGIAFWVRRRAQMAPKRLRLPHIRDDDAPPSHQPLFY.

3.1 Codon Optimisation

In some embodiments, several codons within a parent (e.g., wild-type) HSV gD2 coding sequence are mutated using the method described in WO 2009/049350. In brief, codons of the wild-type coding sequence are replaced with corresponding synonymous codons which are known to have a higher immune response preference than the codons they replace, as set out in Table 1, below:

| First | Synonymous | First | Synonymous | First | Synonymous |
|---|---|---|---|---|---|
| Codon | Codon | Codon | Codon | Codon | Codon |
| Ala^{GCG} | Ala^{GCT} | Ile^{ATA} | Ile^{ATC} | Ser^{AGT} | Ser^{TCG} |
| Ala^{GCG} | Ala^{GCC} | Ile^{ATA} | Ile^{ATT} | Ser^{AGT} | Ser^{TCT} |
| Ala^{GCA} | Ala^{GCT} | Ile^{ATT} | Ile^{ATC} | Ser^{AGT} | Ser^{TCA} |
| Ala^{GCA} | Ala^{GCC} | | | Ser^{AGT} | Ser^{TCC} |
| Ala^{GCC} | Ala^{GCT} | Leu^{TTA} | Leu^{CTG} | Ser^{AGC} | Ser^{TCG} |
| | | Leu^{TTA} | Leu^{CTC} | Ser^{AGC} | Ser^{TCT} |
| Arg^{CGG} | Arg^{CGA} | Leu^{TTA} | Leu^{CTA} | Ser^{AGC} | Ser^{TCA} |
| Arg^{CGG} | Arg^{CGC} | Leu^{TTA} | Leu^{CTT} | Ser^{AGC} | Ser^{TCC} |
| Arg^{CGG} | Arg^{CGT} | Leu^{TTA} | Leu^{TTG} | Ser^{TCC} | Ser^{TCG} |
| Arg^{CGG} | Arg^{AGA} | Leu^{TTG} | Leu^{CTG} | Ser^{TCA} | Ser^{TCG} |
| Arg^{AGG} | Arg^{CGA} | Leu^{TTG} | Leu^{CTC} | Ser^{TCT} | Ser^{TCG} |
| Arg^{AGG} | Arg^{CGC} | Leu^{TTG} | Leu^{CTA} | | |
| Arg^{AGG} | Arg^{CGT} | Leu^{TTG} | Leu^{CTT} | Thr^{ACT} | Thr^{ACG} |
| Arg^{AGG} | Arg^{AGA} | Leu^{CTT} | Leu^{CTG} | Thr^{ACT} | Thr^{ACC} |
| | | Leu^{CTT} | Leu^{CTC} | Thr^{ACT} | Thr^{ACA} |
| Asn^{AAT} | Asn^{AAC} | Leu^{CTA} | Leu^{CTG} | Thr^{ACA} | Thr^{ACG} |
| | | Leu^{CTA} | Leu^{CTC} | Thr^{ACA} | Thr^{ACC} |
| Asp^{GAT} | Asp^{GAC} | | | Thr^{ACC} | Thr^{ACG} |
| | | Phe^{TTC} | Phe^{TTT} | | |
| Cys^{TGT} | Cys^{TGC} | | | Tyr^{TAT} | Tyr^{TAC} |
| | | Pro^{CCG} | Pro^{CCC} | | |
| Glu^{GAG} | Glu^{GAA} | Pro^{CCG} | Pro^{CCT} | Val^{GTA} | Val^{GTG} |
| | | Pro^{CCA} | Pro^{CCC} | Val^{GTA} | Val^{GTC} |
| Gly^{GGC} | Gly^{GGA} | Pro^{CCA} | Pro^{CCT} | Val^{GTA} | Val^{GTT} |
| Gly^{GGT} | Gly^{GGA} | Pro^{CCT} | Pro^{CCC} | Val^{GTT} | Val^{GTG} |
| Gly^{GGG} | Gly^{GGA} | | | Val^{GTT} | Val^{GTC} |

In specific examples, the invention contemplates codon-optimizing coding sequences that encode amino acid sequences corresponding to at least a portion of a wild-type HSV gD2 polypeptide, which involves changing all Ala to GCT; Arg CGG and AGG to CGA and AGA, respectively; Glu to GAA; Gly to GGA; Ile to ATC; all Leu to CTG; Phe to TTT, Pro to CCT or CCC, Ser to TCG, Thr to ACG; and all Val except GTG to GTC. These modifications avoid, with the exception of Leu and Be, changing codons to mammalian consensus-preferred codons. As the codon with the highest immune response preference encoding Leu and Ile amino acids were significantly higher than the alternative synonymous codons, and in light of the frequency of Leu and Be residues in the HSV gD2 polypeptide sequence (39 leucine amino acids and 23 isoleucine amino acids) mammalian consensus-preferred codons were not avoided, to ensure substantial expression of the constructs. An illustrative example of a polynucleotide that accords with such embodiments is as follows:

[SEQ ID NO: 3]
AAGCTTGCCGCCACCATGGGACGTCTGACGTCGGGAGTCGGAACGGCTG
CTCTGCTGGTCGTCGCTGTGGGACTGCGCGTCGTCTGCGCTAAATACGC
TCTGGCTGACCCCTCGCTGAAGATGGCTGATCCCAATCGATTTCGCGGA
AAGAACCTGCCCGTCCTGGACCAGCTGACGGACCCCCCCGGAGTGAAGC
GTGTCTACCACATCCAGCCCTCGCTGGAAGACCCCTTTCAGCCCCCCTC
GATCCCCATCACGGTGTACTACGCTGTGCTGGAACGTGCTTGCCGCTCG
GTGCTGCTGCATGCTCCCTCGGAAGCTCCCCAGATCGTGCGCGGAGCTT
CGGACGAAGCTCGAAAGCACACGTACAACCTGACGATCGCTTGGTATCG
CATGGGAGACAATTGCGCTATCCCCATCACGGTCATGGAATACACGGAA
TGCCCCTACAACAAGTCGCTGGGAGTCTGCCCCATCCGAACGCAGCCCC
GCTGGTCGTACTATGACTCGTTTTCGGCTGTCTCGGAAGATAACCTGGG
ATTTCTGATGCACGCTCCCGCTTTTGAAACGGCTGGAACGTACCTGCGA
CTGGTGAAGATCAACGACTGGACGGAAATCACGCAATTTATCCTGGAAC
ACCGAGCTCGCGCTTCGTGCAAGTACGCTCTGCCCCTGCGCATCCCCCC
CGCTGCTTGCCTGACGTCGAAGGCTTACCAACAGGGAGTGACGGTCGAC
TCGATCGGAATGCTGCCCCGCTTTATCCCCGAAAACCAGCGCACGGTCG
CTCTGTACTCGCTGAAAATCGCTGGATGGCACGGACCCAAGCCCCCCTA
CACGTCGACGCTGCTGCCCCCCGAACTGTCGGACACGACGAACGCTACG
CAACCCGAACTGGTCCCCGAAGACCCCGAAGACTCGGCTCTGCTGGAAG
ATCCCGCTGGAACGGTGTCGTCGCAGATCCCCCCCAACTGGCACATCCC
CTCGATCCAGGACGTCGCTCCCCACCACGCTCCCGCTGCTCCCTCGAAC
CCCGGACTGATCATCGGAGCTCTGGCTGGATCGACGCTGGCTGTGCTGG
TCATCGGAGGAATCGCTTTTTGGGTCCGCCGCCGCGCTCAGATGGCTCC
CAAGCGCCTGCGTCTGCCCCACATCCGAGATGACGACGCTCCCCCCTCG
CACCAGCCCCTGTTTTACTAGCTCGAG.

In some embodiments, the second synthetic coding sequence encodes an amino acid sequence corresponding to at least a portion of a wild-type HSV gD2 polypeptide.

In some embodiments, the second synthetic coding sequence encodes an amino acid sequence corresponding a portion of a wild-type HSV gD2 polypeptide that lacks the gD2 signal peptide and transmembrane domain regions. Although not necessary, removal of these regions ensures that the HSV gD2 polypeptide is not secreted from the cell, thus improving the likelihood of the polypeptide being degraded and eliciting a cellular immune response. For example, the synthetic coding sequence may encode amino acids 25-331 of the wild-type HSV gD2 amino acid sequence. In an illustrative example of this type, the second synthetic coding sequence comprises the following sequence:

[SEQ ID NO: 4]
AAGCTTGCCGCCACCATGCAGATCTTTGTGAAGACGCTGACGGGAAAGAC
GATCACGCTGGAAGTGGAACCCTCGGACACGATCGAAAACGTGAAGGCTA
AGATCCAGGACAAGGAAGGAATCCCCCCCGACCAGCAGAGACTGATCTTT
GCTGGAAAGCAGCTGGAAGACGGACGCACGCTGTCGGACTACAACATCCA
GAAGGAATCGACGCTGCACCTGGTGCTGAGACTGCGCGGAGCTGCTAAAT
ACGCTCTGGCTGACCCCTCGCTTAAGATGGCTGATCCCAATCGATTTCGC
GGAAAGAACCTGCCCGTCCTGGACCAGCTGACGGACCCCCCCGGAGTGAA
GCGTGTCTACCACATCCAGCCCTCGCTGGAAGACCCCTTTCAGCCCCCCT
CGATCCCCATCACGGTGTACTACGCTGTGCTGGAACGTGCTTGCCGCTCG
GTGCTGCTGCATGCTCCCTCGGAAGCTCCCCAGATCGTGCGCGGAGCTTC
GGACGAAGCTCGAAAGCACACGTACAACCTGACGATCGCTTGGTATCGCA
TGGGAGACAATTGCGCTATCCCCATCACGGTCATGGAATACACGGAATGC
CCCTACAACAAGTCGCTGGGAGTCTGCCCCATCCGAACGCAGCCCCGCTG
GTCGTACTATGACTCGTTTTCGGCTGTCTCGGAAGATAACCTGGGATTTC
TGATGCACGCTCCCGCTTTTGAAACGGCTGGAACGTACCTGCGACTGGTG
AAGATCAACGACTGGACGGAAATCACGCAATTTATCCTGGAACACCGAGC
TCGCGCTTCGTGCAAGTACGCTCTGCCCCTGCGCATCCCCCCCGCTGCTT
GCCTGACGTCGAAGGCTTACCAACAGGGAGTGACGGTCGACTCGATCGGA
ATGCTGCCCCGCTTTATCCCCGAAAACCAGCGCACGGTCGCTCTGTACTC
GCTGAAAATCGCTGGATGGCACGGACCCAAGCCCCCCTACACGTCGACGC
TGCTGCCCCCCGAACTGTCGGACACGACGAACGCTACGCAACCCGAACTG
GTCCCCGAAGACCCCGAAGACTCGGCTCTGCTGGAAGATCCCGCTGGAAC
GGTGTCGTCGCAGATCCCCCCCAACTGGCACATCCCCTCGATCCAGGACG
TCGCTCCCCACCACTAGCTCGAG.

The parent HSV gD2 coding sequence that is codon-optimized to make the synthetic coding sequence is suitably a wild-type or natural gene. However, it is possible that the parent HSV gD2 coding sequence is not naturally-occurring but has been engineered using recombinant techniques. Wild-type polynucleotides can be obtained from any suitable source, such as from eukaryotic or prokaryotic organisms, including but not limited to mammals or other animals, and pathogenic organisms such as yeasts, bacteria, protozoa and viruses.

As will be appreciated by those of skill in the art, it is generally not necessary to immunize with a synthetic coding sequence encoding a polypeptide that shares exactly the same amino acid sequence with an HSV gD2 polypeptide to produce an immune response to that antigen. In some embodiments, therefore, the polypeptide encoded by the synthetic coding sequence is a variant of at least a portion of an HSV gD2 polypeptide. “Variant” polypeptides include proteins derived from the HSV gD2 polypeptide by deletion (so-called truncation) or addition of one or more amino acids to the N-terminal and/or C-terminal end of the HSV gD2 polypeptide; deletion or addition of one or more amino acids at one or more sites in the HSV gD2 polypeptide; or substitution of one or more amino acids at one or more sites in the HSV gD2 polypeptide. Variant polypeptides encompassed by the present invention will have at least 40%, 50%, 60%, 70%, generally at least 75%, 80%, 85%, typically at least about 90% to 95% or more, and more typically at least about 96%, 97%, 98%, 99% or more sequence similarity or identity with the amino acid sequence of a wild-type HSV gD2 polypeptide or portion thereof as determined by sequence alignment programs described elsewhere herein using default parameters. A variant of an HSV gD2 polypeptide may differ from the wild-type sequence generally by as much 200, 100, 50 or 20 amino acid residues or suitably by as few as 1-15 amino acid residues, as few as 1-10, such as 6-10, as few as 5, as few as 4, 3, 2, or even 1 amino acid residue.

Variant polypeptides corresponding to at least a portion of an HSV gD2 polypeptide may contain conservative amino acid substitutions at various locations along their sequence, as compared to the HSV gD2 polypeptide sequence. A “conservative amino acid substitution” is one in which the amino acid residue is replaced with an amino acid residue having a similar side chain. Families of amino acid residues having similar side chains have been defined in the art, which can be generally sub-classified as follows:

Acidic: The residue has a negative charge due to loss of H ion at physiological pH and the residue is attracted by aqueous solution so as to seek the surface positions in the conformation of a peptide in which it is contained when the peptide is in aqueous medium at physiological pH. Amino acids having an acidic side chain include glutamic acid and aspartic acid.

Basic: The residue has a positive charge due to association with H ion at physiological pH or within one or two pH units thereof (e.g., histidine) and the residue is attracted by aqueous solution so as to seek the surface positions in the conformation of a peptide in which it is contained when the peptide is in aqueous medium at physiological pH. Amino acids having a basic side chain include arginine, lysine and histidine.

Charged: The residues are charged at physiological pH and, therefore, include amino acids having acidic or basic side chains (i.e., glutamic acid, aspartic acid, arginine, lysine and histidine).

Hydrophobic: The residues are not charged at physiological pH and the residue is repelled by aqueous solution so as to seek the inner positions in the conformation of a peptide in which it is contained when the peptide is in aqueous medium. Amino acids having a hydrophobic side chain include tyrosine, valine, isoleucine, leucine, methionine, phenylalanine and tryptophan.

Neutral/polar: The residues are not charged at physiological pH, but the residue is not sufficiently repelled by aqueous solutions so that it would seek inner positions in the conformation of a peptide in which it is contained when the peptide is in aqueous medium. Amino acids having a neutral/polar side chain include asparagine, glutamine, cysteine, histidine, serine and threonine.

This description also characterizes certain amino acids as “small” since their side chains are not sufficiently large, even if polar groups are lacking, to confer hydrophobicity. With the exception of proline, “small” amino acids are those with four carbons or less when at least one polar group is on the side chain and three carbons or less when not. Amino acids having a small side chain include glycine, serine, alanine and threonine. The gene-encoded secondary amino acid proline is a special case due to its known effects on the secondary conformation of peptide chains. The structure of proline differs from all the other naturally-occurring amino acids in that its side chain is bonded to the nitrogen of the α-amino group, as well as the α-carbon. Several amino acid similarity matrices (e.g., PAM120 matrix and PAM250 matrix as disclosed for example by Dayhoff et al. (1978) A model of evolutionary change in proteins. Matrices for determining distance relationships In M. O. Dayhoff, (ed.), Atlas of protein sequence and structure, Vol. 5, pp. 345-358, National Biomedical Research Foundation, Washington D.C.; and by Gonnet et al., 1992, Science 256(5062): 144301445), however, include proline in the same group as glycine, serine, alanine and threonine. Accordingly, for the purposes of the present invention, proline is classified as a “small” amino acid.

The degree of attraction or repulsion required for classification as polar or nonpolar is arbitrary and, therefore, amino acids specifically contemplated by the invention have been classified as one or the other. Most amino acids not specifically named can be classified on the basis of known behavior.

Amino acid residues can be further sub-classified as cyclic or noncyclic, and aromatic or nonaromatic, self-explanatory classifications with respect to the side-chain substituent groups of the residues, and as small or large. The residue is considered small if it contains a total of four carbon atoms or less, inclusive of the carboxyl carbon, provided an additional polar substituent is present; three or less if not. Small residues are, of course, always nonaromatic. Dependent on their structural properties, amino acid residues may fall in two or more classes. For the naturally-occurring protein amino acids, sub-classification according to the this scheme is presented in the Table 3.

TABLE 3

| Original Residue | Exemplary Substitutions |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile, |
| Phe | Met, Leu, Tyr |
| Ser | Thr |
| Thr | Ser |
| Trp | Tyr |
| Tyr | Trp, Phe |
| Val | Ile, Leu |

Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting variant polypeptide. Conservative substitutions are shown in Table 4 below under the heading of exemplary substitutions. More preferred substitutions are shown under the heading of preferred substitutions. Amino acid substitutions falling within the scope of the invention, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. After the substitutions are introduced, the variants are screened for biological activity.

TABLE 4
EXEMPLARY AND PREFERRED
AMINO ACID SUBSTITUTIONS

| | | Preferred |
|---|---|---|
| Original Residue | Exemplary Substitutions | Substitutions |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn, His, Lys, | Asn |
| Glu | Asp, Lys | Asp |
| Gly | Pro | Pro |
| His | Asn, Gln, Lys, Arg | Arg |
| Ile | Leu, Val, Met, Ala, Phe, | Leu |
| | Norleu | |
| Leu | Norleu, Ile, Val, Met, Ala, Phe | Ile |
| Lys | Arg, Gln, Asn | Arg |
| Met | Leu, Ile, Phe | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Norleu | Leu |

Alternatively, similar amino acids for making conservative substitutions can be grouped into three categories based on the identity of the side chains. The first group includes glutamic acid, aspartic acid, arginine, lysine, histidine, which all have charged side chains; the second group includes glycine, serine, threonine, cysteine, tyrosine, glutamine, asparagine; and the third group includes leucine, isoleucine, valine, alanine, proline, phenylalanine, tryptophan, methionine, as described in Zubay, G., Biochemistry, third edition, Wm.C. Brown Publishers (1993).

3.2 Methods of Substituting Codons

Replacement of one codon for another can be achieved using standard methods known in the art. For example, codon modification of a parent polynucleotide can be effected using several known mutagenesis techniques including, for example, oligonucleotide-directed mutagenesis, mutagenesis with degenerate oligonucleotides, and region-specific mutagenesis. Exemplary in vitro mutagenesis techniques are described for example in U.S. Pat. Nos. 4,184,917, 4,321,365 and 4,351,901 or in the relevant sections of Ausubel, et al. (CURRENT PROTOCOLS IN MOLECULAR BIOLOGY, John Wiley & Sons, Inc. 1997) and of Sambrook, et al., (MOLECULAR CLONING. A LABORATORY MANUAL, Cold Spring Harbor Press, 1989). Instead of in vitro mutagenesis, the synthetic coding sequence can be synthesized de novo using readily available machinery as described, for example, in U.S. Pat. No. 4,293,652. However, it should be noted that the present invention is not dependent on, and not directed to, any one particular technique for constructing the synthetic coding sequence.

### 4. Synthetic Constructs

4.1 Regulatory Nucleic Acids

The present invention further contemplates first and second constructs each comprising a synthetic coding sequences that is operably linked to a regulatory nucleic acid. The regulatory nucleic acid suitably comprises transcriptional and/or translational control sequences, which will be compatible for expression in the organism of interest or in cells of that organism. Typically, the transcriptional and translational regulatory control sequences include, but are not limited to, a promoter sequence, a 5′ non-coding region, a cis-regulatory region such as a functional binding site for transcriptional regulatory protein or translational regulatory protein, an upstream open reading frame, ribosomal-binding sequences, transcriptional start site, translational start site, and/or nucleotide sequence which encodes a leader sequence, termination codon, translational stop site and a 3′ non-translated region. Constitutive or inducible promoters as known in the art are contemplated by the invention. The promoters may be either naturally occurring promoters, or hybrid promoters that combine elements of more than one promoter. Promoter sequences contemplated by the present invention may be native to the organism of interest or may be derived from an alternative source, where the region is functional in the chosen organism. The choice of promoter will differ depending on the intended host or cell or tissue type. For example, promoters which could be used for expression in mammals include the metallothionein promoter, which can be induced in response to heavy metals such as cadmium, the β-actin promoter as well as viral promoters such as the SV40 large T antigen promoter, human cytomegalovirus (CMV) immediate early (IE) promoter, Rous sarcoma virus LTR promoter, the mouse mammary tumor virus LTR promoter, the adenovirus major late promoter (Ad MLP), the herpes simplex virus promoter, and a HPV promoter, particularly the HPV upstream regulatory region (URR), among others. All these promoters are well described and readily available in the art.

Enhancer elements may also be used herein to increase expression levels of the mammalian constructs. Examples include the SV40 early gene enhancer, as described for example in Dijkema et al. (1985, EMBO J. 4:761), the enhancer/promoter derived from the long terminal repeat (LTR) of the Rous Sarcoma Virus, as described for example in Gorman et al., (1982, Proc. Natl. Acad. Sci. USA 79:6777) and elements derived from human CMV, as described for example in Boshart et al. (1985, Cell 41:521), such as elements included in the CMV intron A sequence.

The first and second constructs may also comprise a 3′ non-translated sequence. A 3′ non-translated sequence refers to that portion of a gene comprising a DNA segment that contains a polyadenylation signal and any other regulatory signals capable of effecting mRNA processing or gene expression. The polyadenylation signal is characterized by effecting the addition of polyadenylic acid tracts to the 3′ end of the mRNA precursor. Polyadenylation signals are commonly recognized by the presence of homology to the canonical form 5′ AATAAA-3′ although variations are not uncommon. The 3′ non-translated regulatory DNA sequence preferably includes from about 50 to 1,000 nts and may contain transcriptional and translational termination sequences in addition to a polyadenylation signal and any other regulatory signals capable of effecting mRNA processing or gene expression.

In some embodiments, the first and second constructs further contain a selectable marker gene to permit selection of cells containing the construct. Selection genes are well known in the art and will be compatible for expression in the cell of interest.

It will be understood, however, that expression of protein-encoding polynucleotides in heterologous systems is now well known, and the present invention is not necessarily directed to or dependent on any particular vector, transcriptional control sequence or technique for expression of the polynucleotides. Rather, synthetic coding sequences prepared according to the methods set forth herein may be introduced into a mammal in any suitable manner in the form of any suitable construct or vector, and the synthetic coding sequences may be expressed with known transcription regulatory elements in any conventional manner.

Furthermore, the first and second constructs can be constructed to include chimeric antigen-coding gene sequences, encoding, e.g., multiple antigens/epitopes of interest, for example derived from a single or from more than one HSV gD2 polypeptide. In certain embodiments, multi-cistronic cassettes (e.g., bi-cistronic cassettes) can be constructed allowing expression of multiple adjuvants and/or antigenic polypeptides from a single mRNA using, for example, the EMCV IRES, or the like. In other embodiments, adjuvants and/or antigenic polypeptides can be encoded on separate coding sequences that are operably connected to independent transcription regulatory elements.

4.2 Protein Adjuvants and Protein-Destabilising Elements

In addition, the first and second constructs can be constructed to include sequences coding for protein adjuvants. Particularly suitable are detoxified mutants of bacterial ADP-ribosylating toxins, for example, diphtheria toxin, pertussis toxin (PT), cholera toxin (CT), Escherichia coli heat-labile toxins (LT1 and LT2), Pseudomonas endotoxin A, Clostridium botulinum C2 and C3 toxins, as well as toxins from C. perfringens, C. spiriforma and C. difficile. In some embodiments, the first and second constructs include coding sequences for detoxified mutants of E. coli heat-labile toxins, such as the LT-K63 and LT-R72 detoxified mutants, described in U.S. Pat. No. 6,818,222.

In some embodiments, the adjuvant is a protein-destabilising element, which increases processing and presentation of the polypeptide that corresponds to at least a portion of the HSV gD2 polypeptide through the class I MHC pathway, thereby leading to enhanced cell-mediated immunity against the polypeptide. Illustrative protein-destabilising elements include intracellular protein degradation signals or degrons which may be selected without limitation from a destabilising amino acid at the amino-terminus of a polypeptide of interest, a PEST region or a ubiquitin. For example, the coding sequence for the polypeptide can be modified to include a destabilising amino acid at its amino-terminus so that the protein so modified is subject to the N-end rule pathway as disclosed, for example, by Bachmair et al. in U.S. Pat. No. 5,093,242 and by Varshaysky et al. in U.S. Pat. No. 5,122,463. In some embodiments, the destabilising amino acid is selected from isoleucine and glutamic acid, especially from histidine tyrosine and glutamine, and more especially from aspartic acid, asparagine, phenylalanine, leucine, tryptophan and lysine. In certain embodiments, the destabilising amino acid is arginine. In some proteins, the amino-terminal end is obscured as a result of the protein's conformation (i.e., its tertiary or quaternary structure). In these cases, more extensive alteration of the amino-terminus may be necessary to make the protein subject to the N-end rule pathway. For example, where simple addition or replacement of the single amino-terminal residue is insufficient because of an inaccessible amino-terminus, several amino acids (including lysine, the site of ubiquitin joining to substrate proteins) may be added to the original amino-terminus to increase the accessibility and/or segmental mobility of the engineered amino terminus. In some embodiments, a nucleic acid sequence encoding the amino-terminal region of the polypeptide can be modified to introduce a lysine residue in an appropriate context. This can be achieved most conveniently by employing DNA constructs encoding “universal destabilising segments”. A universal destabilising segment comprises a nucleic acid construct which encodes a polypeptide structure, preferably segmentally mobile, containing one or more lysine residues, the codons for lysine residues being positioned within the construct such that when the construct is inserted into the coding sequence of the protein-encoding synthetic coding sequence, the lysine residues are sufficiently spatially proximate to the amino-terminus of the encoded protein to serve as the second determinant of the complete amino-terminal degradation signal. The insertion of such constructs into the 5′ portion of a polypeptide-encoding synthetic coding sequence would provide the encoded polypeptide with a lysine residue (or residues) in an appropriate context for destabilization. In other embodiments, the polypeptide is modified to contain a PEST region, which is rich in an amino acid selected from proline, glutamic acid, serine and threonine, which region is optionally flanked by amino acids comprising electropositive side chains. In this regard, it is known that amino acid sequences of proteins with intracellular half-lives less than about 2 hours contain one or more regions rich in proline (P), glutamic acid (E), serine (S), and threonine (T) as for example shown by Rogers et al. (1986, Science 234 (4774): 364-368). In still other embodiments, the polypeptide is conjugated to a ubiquitin or a biologically active fragment thereof, to produce a modified polypeptide whose rate of intracellular proteolytic degradation is increased, enhanced or otherwise elevated relative to the unmodified polypeptide.

One or more adjuvant polypeptides may be co-expressed with an ‘antigenic’ polypeptide that corresponds to at least a portion of the HSV gD2 polypeptide. In certain embodiments, adjuvant and antigenic polypeptides may be co-expressed in the form of a fusion protein comprising one or more adjuvant polypeptides and one or more antigenic polypeptides. Alternatively, adjuvant and antigenic polypeptides may be co-expressed as separate proteins.

4.3 Vectors

The first and second constructs described above are suitably in the form of a vector that is suitable for expression of recombinant proteins in mammalian cells, and particularly those identified for the induction of neutralizing immune responses by genetic immunization. Vectors prepared specifically for use in DNA vaccines generally combine a eukaryotic region that directs expression of the transgene in the target organism with a bacterial region that provides selection and propagation in the Escherichia coli (E. coli) host. The eukaryotic region contains a promoter upstream, and a polyadenylation signal (polyA) downstream, of the gene of interest. Upon transfection into the cell nucleus, the promoter directs transcription of an mRNA that includes the transgene. The polyadenylation signal mediates mRNA cleavage and polyadenylation, which leads to efficient mRNA export to the cytoplasm. A Kozak sequence (gccgccRccATGG consensus, transgene ATG start codon within the Kozak sequence is underlined, critical residues in caps, R=A or G) is often included. The Kozak sequence is recognized in the cytoplasm by ribosomes and directs efficient transgene translation. The constitutive human Cytomegalovirus (CMV) promoter is the most common promoter used in DNA vaccines since it is highly active in most mammalian cells transcribing higher levels of mRNA than alternative viral or cellular promoters. PolyA signals are typically used to increase polyadenylation efficiency resulting in increased mRNA levels, and improved transgene expression.

In some embodiments, the vector comprises a first or second synthetic coding sequence without any additional and/or non-functional sequences, (e.g., cryptic ORFs that may be expressed in the subject). This is especially beneficial within the transcribed UTRs to prevent production of vector encoded cryptic peptides in a subject that may induce undesirable adaptive immune responses. Illustrative examples of vectors that are suitable for use with the present invention include NTC8485 and NCT8685 (Nature Technology Corporation, Nebraska, USA). Alternatively, the parent vector, NTC7485, can be used. NTC7485 was designed to comply with the U.S. Food and Drug Administration (FDA) regulatory guidance regarding DNA vaccine vector compositions (FDA 1996, FDA 2007, and reviewed in Williams et al, 2009). Specifically, all sequences that are not essential for Escherichia coli plasmid replication or mammalian cell expression of the target gene were eliminated. Synthetic eukaryotic mRNA leader and terminator sequences were utilized in the vector design to limit DNA sequence homology with the human genome in order to reduce the possibility of chromosomal integration.

In other embodiments, the vector may comprise a nucleic acid sequence encoding an ancillary functional sequence (e.g., a sequence effecting transport or post translational sequence modification of HSV gD2 polypeptide, non-limiting examples of which include a signal or targeting sequence). For example, NTC8482 targets encoded protein into the secretory pathway using an optimized tissue plasminogen activator (TPA) signal peptide.

In some embodiments, expression of the HSV gD2 antigen is driven from an optimized chimeric promoter-intron (e.g., SV40-CMV-HTLV-1 R synthetic intron). In one aspect of these embodiments, the vectors encode a consensus Kozak translation initiation sequence and an ATG start codon. Notably, the chimeric cytomegalovirus (CMV) promoter achieves significantly higher expression levels than traditional human CMV promoter-based vectors (Luke et al, 2009).

In one embodiment, the DNA plasmid is cloned into the NTC8485, NTC8685, or NTC9385R vector families, which combine minimal prokaryotic sequences and include an antibiotic free sucrose selectable marker. These families also contain a novel chimeric promoter that directs superior mammalian cell expression (see, Luke et al., 2009; Luke et al, 2011; and Williams, 2013).

4.4 Antibiotic-Free Selection Using RNA Selection Markers

As described above, in some embodiments, the vector is free of any non-essential sequences for expressing the synthetic constructs of the invention, for example, an antibiotic-resistance marker. Kanamycin resistance (KanR) is the most utilized resistance gene in vectors to allow selective retention of plasmid DNA during bacterial fermentation. However, to ensure safety regulatory agencies generally recommend elimination of antibiotic-resistance markers from therapeutic and vaccine plasmid DNA vectors. The presence of an antibiotic resistance gene in the vaccine vector is therefore considered undesirable by regulatory agencies, due to the potential transfer of antibiotic resistance to endogenous microbial flora and the potential activation and transcription of the genes from mammalian promoters after cellular incorporation into the genome. Vectors that are retrofit to replace the KanR marker with short RNA antibiotic-free markers generally have the unexpected benefit of improved expression. The NTC7485 vector comprises a kanamycin resistance antibiotic selection marker.

In some embodiments, selection techniques other than antibiotic resistance are used. By way of an illustrative example, the NTC8485, NTC8684 and NTC9385R vectors are derived from the NTC7485 vector, wherein the KanR antibiotic selection marker is replaced with a sucrose selectable RNA-OUT marker. Accordingly, in some embodiments, the vaccine vector comprises an antibiotic-free selection system. Although a number of antibiotic-free plasmid retention systems have been developed in which the vector-encoded selection marker is not protein based, superior expression and manufacture has been observed with SNA vaccine vectors that incorporate RNA based antibiotic-free selection markers.

An illustrative example of a suitable RNA based antibiotic-free selection system is the sucrose selection vector, RNA-OUT, a small 70 bp antisense RNA system (Nature Technology Corporation, Nebraska, USA); pFAR4 and pCOR vectors encode a nonsense suppressor tRNA marker; and the pMINI vector utilizes the ColE1 origin-encoded RNAI antisense RNA. Each of these plasmid-borne RNAs regulate the translation of a host chromosome encoded selectable marker allowing plasmid selection. For example, RNA-OUT represses expression of a counter-selectable marker (SacB) from the host chromosome (selection host DH5α att_{λ}::P_{5/6 6/6}-RNA-IN-SacB, catR). SacB encodes a levansucrase, which is toxic in the presence of sucrose. Plasmid selection is achieved in the presence of sucrose. Moreover, for both RNA-OUT vectors and pMINI, high yielding fermentation processes have been developed. In all these vectors, replacement of the KanR antibiotic selection marker results has previously been demonstrated to improve transgene expression in the target organism, showing that elimination of antibiotic selection to meet regulatory criteria may unexpectedly also improve vector performance.

4.5 Viral Vectors

In some embodiments, the first and second constructs of the invention are in the form of expression vectors which are suitably selected from self-replicating extrachromosomal vectors (e.g., plasmids) and vectors that integrate into a host genome. In illustrative examples of this type, the expression vectors are viral vectors, such as simian virus 40 (SV40) or bovine papilloma virus (BPV), which has the ability to replicate as extrachromosomal elements (Eukaryotic Viral Vectors, Cold Spring Harbor Laboratory, Gluzman ed., 1982; Sarver et al., 1981, Mol. Cell. Biol. 1:486). Viral vectors include retroviral (lentivirus), adeno-associated virus (see, e.g., Okada, 1996, Gene Ther. 3:957-964; Muzyczka, 1994, J. Clin. Invst. 94:1351; U.S. Pat. Nos. 6,156,303; 6,143,548 5,952,221, describing AAV vectors; see also U.S. Pat. Nos. 6,004,799; 5,833,993), adenovirus (see, e.g., U.S. Pat. Nos. 6,140,087; 6,136,594; 6,133,028; 6,120,764), reovirus, herpesvirus, rotavirus genomes etc., modified for introducing and directing expression of a polynucleotide or transgene in cells. Retroviral vectors can include those based upon murine leukaemia virus (see, e.g., U.S. Pat. No. 6,132,731), gibbon ape leukaemia virus (see, e.g., U.S. Pat. No. 6,033,905), simian immuno-deficiency virus, human immuno-deficiency virus (see, e.g., U.S. Pat. No. 5,985,641), and combinations thereof.

Vectors also include those that efficiently deliver genes to animal cells in vivo (e.g., stem cells) (see, e.g., U.S. Pat. Nos. 5,821,235 and 5,786,340; Croyle et al., 1998, Gene Ther. 5:645; Croyle et al., 1998, Pharm. Res. 15:1348; Croyle et al., 1998, Hum. Gene Ther. 9:561; Foreman et al., 1998, Hum. Gene Ther. 9:1313; Wirtz et al., 1999, Gut 44:800). Adenoviral and adeno-associated viral vectors suitable for in vivo delivery are described, for example, in U.S. Pat. Nos. 5,700,470, 5,731,172 and 5,604,090. Additional vectors suitable for in vivo delivery include herpes simplex virus vectors (see, e.g., U.S. Pat. No. 5,501,979), retroviral vectors (see, e.g., U.S. Pat. Nos. 5,624,820, 5,693,508 and 5,674,703; and WO92/05266 and WO92/14829), bovine papilloma virus (BPV) vectors (see, e.g., U.S. Pat. No. 5,719,054), CMV-based vectors (see, e.g., U.S. Pat. No. 5,561,063) and parvovirus, rotavirus and Norwalk virus vectors. Lentiviral vectors are useful for infecting dividing as well as non-dividing cells (see, e.g., U.S. Pat. No. 6,013,516).

Additional viral vectors which will find use for delivering the nucleic acid molecules encoding the antigens of interest include those derived from the pox family of viruses, including vaccinia virus and avian poxvirus. By way of example, vaccinia virus recombinants expressing the first and second constructs can be constructed as follows. The antigen coding sequence is first inserted into an appropriate vector so that it is adjacent to a vaccinia promoter and flanking vaccinia DNA sequences, such as the sequence encoding thymidine kinase (TK). This vector is then used to transfect cells that are simultaneously infected with vaccinia. Homologous recombination serves to insert the vaccinia promoter plus the gene encoding the coding sequences of interest into the viral genome. The resulting TK-recombinant can be selected by culturing the cells in the presence of 5-bromodeoxyuridine and picking viral plaques resistant thereto.

Alternatively, avipoxviruses, such as the fowlpox and canarypox viruses, can also be used to deliver the genes. Recombinant avipox viruses, expressing immunogens from mammalian pathogens, are known to confer protective immunity when administered to non-avian species. The use of an avipox vector is particularly desirable in human and other mammalian species since members of the avipox genus can only productively replicate in susceptible avian species and therefore are not infective in mammalian cells. Methods for producing recombinant avipoxviruses are known in the art and employ genetic recombination, as described above with. respect to the production of vaccinia viruses. See, e.g., WO 91/12882; WO 89/03429; and WO 92/03545.

Molecular conjugate vectors, such as the adenovirus chimeric vectors described in Michael et al., J. Biol. Chem. (1993) 268:6866-6869 and Wagner et al., Proc. Natl. Acad. Sci. USA (1992) 89:6099-6103, can also be used for gene delivery.

Members of the Alphavirus genus, such as, but not limited to, vectors derived from the Sindbis virus (SIN), Semliki Forest virus (SFV), and Venezuelan Equine Encephalitis virus (VEE), will also find use as viral vectors for delivering the first and second constructs of the present invention. For a description of Sindbis-virus derived vectors useful for the practice of the instant methods, see, Dubensky et al. (1996, J. Virol. 70:508-519; and International Publication Nos. WO 95/07995, WO 96/17072); as well as, Dubensky, Jr., T. W., et al., U.S. Pat. No. 5,843,723, and Dubensky, Jr., T. W., U.S. Pat. No. 5,789,245. Exemplary vectors of this type are chimeric alphavirus vectors comprised of sequences derived from Sindbis virus and Venezuelan equine encephalitis virus. See, e.g., Perri et al. (2003, J. Virol. 77: 10394-10403) and International Publication Nos. WO 02/099035, WO 02/080982, WO 01/81609, and WO 00/61772.

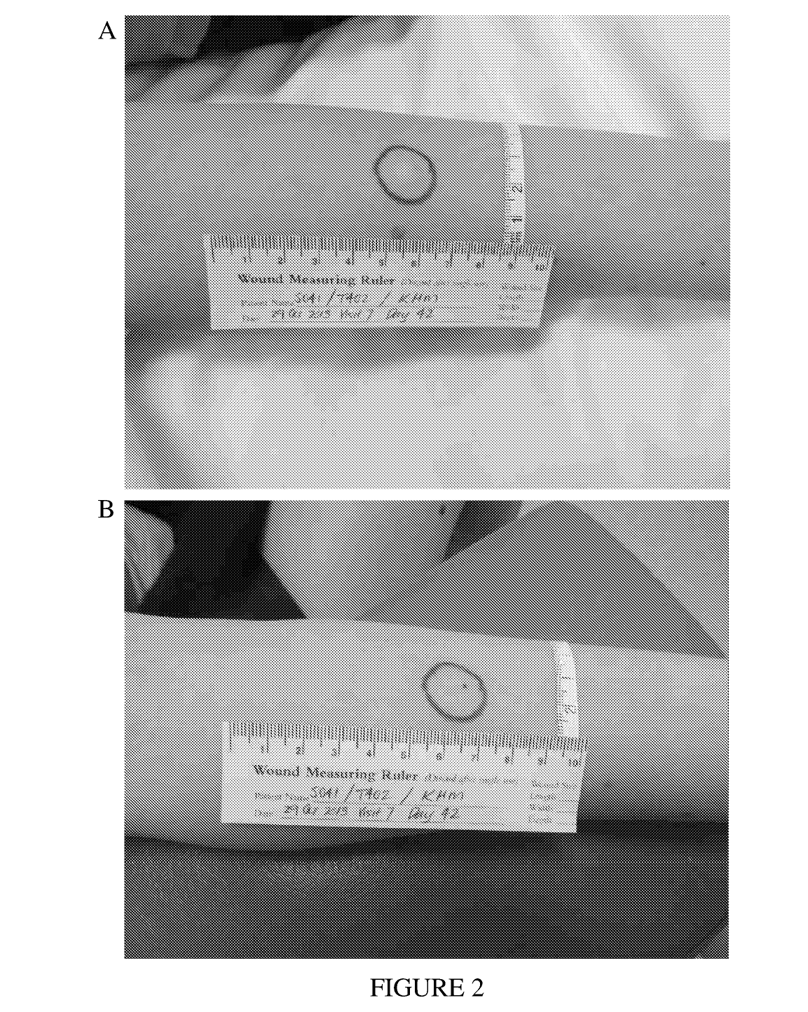

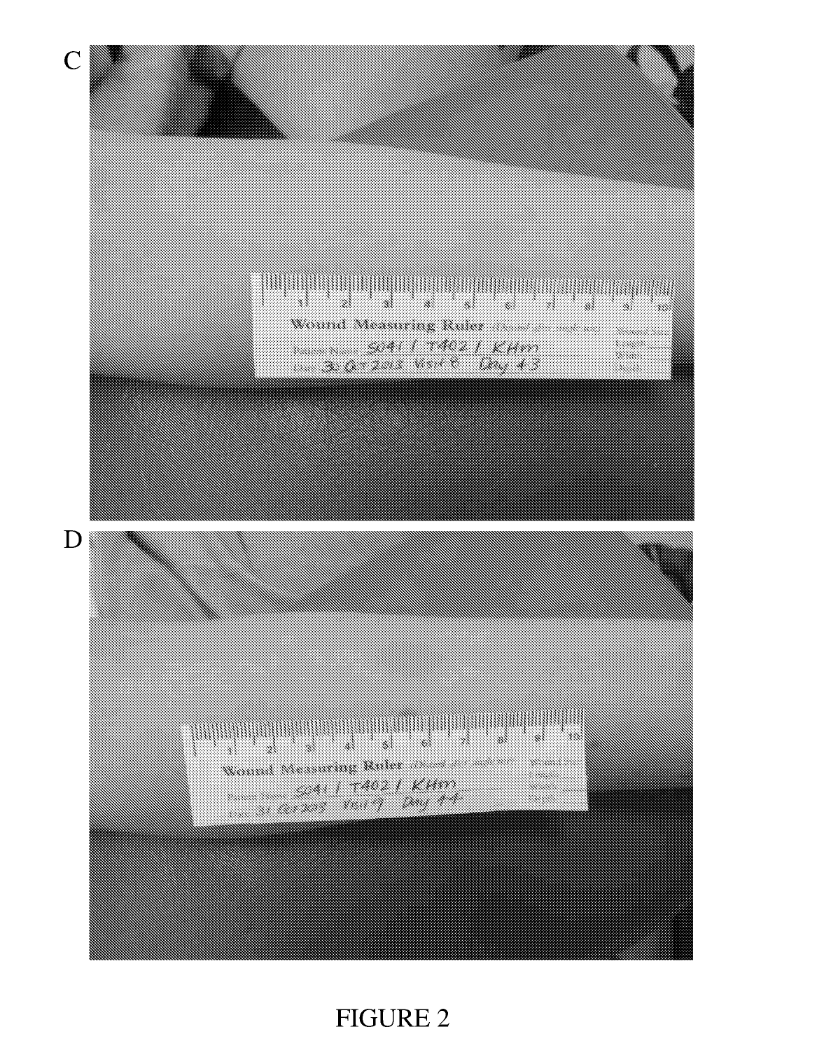

In other illustrative embodiments, lentiviral vectors are employed to deliver the first and second constructs of the invention into selected cells or tissues. Typically, these vectors comprise a 5′ lentiviral LTR, a tRNA binding site, a packaging signal, a promoter operably linked to one or more genes of interest, an origin of second strand DNA synthesis and a 3′ lentiviral LTR, wherein the lentiviral vector contains a nuclear transport element. The nuclear transport element may be located either upstream (5′) or downstream (3′) of a coding sequence of interest (for example, a synthetic Gag or Env expression cassette of the present invention). A wide variety of lentiviruses may be utilized within the context of the present invention, including for example, lentiviruses selected from the group consisting of HIV, HIV-1, HIV-2, FIV, BIV, EIAV, MVV, CAEV, and SIV. Illustrative examples of lentiviral vectors are described in PCT Publication Nos. WO 00/66759, WO 00/00600, WO 99/24465, WO 98/51810, WO 99/51754, WO 99/31251, WO 99/30742, and WO 99/15641. Desirably, a third generation SIN lentivirus is used. Commercial suppliers of third generation SIN (self-inactivating) lentiviruses include Invitrogen (ViraPower Lentiviral Expression System). Detailed methods for construction, transfection, harvesting, and use of lentiviral vectors are given, for example, in the Invitrogen technical manual “ViraPower Lentiviral Expression System version B 050102 25-0501”, available at http://www.invitrogen.com/Content/Tech-Online/molecular_biology/manuals_p-ps/virapower_lentiviral_system_man.pdf. Lentiviral vectors have emerged as an efficient method for gene transfer. Improvements in biosafety characteristics have made these vectors suitable for use at biosafety level 2 (BL2). A number of safety features are incorporated into third generation SIN (self-inactivating) vectors. Deletion of the viral 3′ LTR U3 region results in a provirus that is unable to transcribe a full length viral RNA. In addition, a number of essential genes are provided in trans, yielding a viral stock that is capable of but a single round of infection and integration. Lentiviral vectors have several advantages, including: 1) pseudotyping of the vector using amphotropic envelope proteins allows them to infect virtually any cell type; 2) gene delivery to quiescent, post mitotic, differentiated cells, including neurons, has been demonstrated; 3) their low cellular toxicity is unique among transgene delivery systems; 4) viral integration into the genome permits long term transgene expression; 5) their packaging capacity (6-14 kb) is much larger than other retroviral, or adeno-associated viral vectors. In a recent demonstration of the capabilities of this system, lentiviral vectors expressing GFP were used to infect murine stem cells resulting in live progeny, germline transmission, and promoter-, and tissue-specific expression of the reporter (Ailles, L. E. and Naldini, L., HIV-1-Derived Lentiviral Vectors. In: Trono, D. (Ed.), Lentiviral Vectors, Springer-Verlag, Berlin, Heidelberg, New York, 2002, pp. 31-52). An example of the current generation vectors is outlined in FIG. 2 of a review by Lois et al. (2002, Science, 295 868-872).

The first and second constructs can also be delivered without a vector. For example, the constructs can be packaged as DNA or RNA in liposomes prior to delivery to the subject or to cells derived therefrom. Lipid encapsulation is generally accomplished using liposomes which are able to stably bind or entrap and retain nucleic acid. The ratio of condensed DNA to lipid preparation can vary but will generally be around 1:1 (mg DNA:micromoles lipid), or more of lipid. For a review of the use of liposomes as carriers for delivery of nucleic acids, see, Hug and Sleight, (1991, Biochim. Biophys. Acta. 1097:1-17); and Straubinger et al., in Methods of Enzymology (1983), Vol. 101, pp. 512-527.

In other embodiments, the first and second constructs comprise, consist or consist essentially of an mRNA coding sequence comprising an HSV gD2 coding sequence. The HSV gD2 coding sequence may optionally comprise a Kozak sequence and/or a polyadenylated sequence, as described above. Suitably, the first and second constructs optionally further comprise chemical modification to the RNA structure as known in the art, such as phosphorothioation of the backbone or 2′-methoxyethylation (2′MOE) of ribose sugar groups to enhance uptake, stability, and ultimate effectiveness of the mRNA coding sequence (see, Agrawal 1999; Gearry et al, 2001).

4.6 Minicircle Vectors

In some embodiments, the first and/or second constructs are in the form of minicircle vectors. A minicircle vector is a small, double stranded circular DNA molecule that provides for persistent, high level expression of an HSV gD2 coding sequence that is present on the vector, which sequence of interest may encode a polypeptide (e.g., a HSV gD2 polypeptide). The HSV gD2 coding sequence is operably linked to regulatory sequences present on the minicircle vector, which regulatory sequences control its expression. Suitable minicircle vectors for use with the present invention are described, for example, in published U.S. Patent Application No. 2004/0214329, and can be prepared by the method described in Darquet et al, Gene Ther. (1997) 4: 1341-1349. In brief, an HSV gD2 coding sequence is flanked by attachment sites for a recombinase, which is expressed in an inducible fashion in a portion of the vector sequence outside of the coding sequence.

In brief, minicircle vectors can be prepared with plasmids similar to pBAD..phi.C31.hFIX and pBAD..phi.C31.RHB and used to transform E. coli. Recombinases known in the art, for example, lambda and cre, are suitable for incorporation to the minicircle vectors. The expression cassettes present in the minicircle vectors may contain sites for transcription initiation and termination, as well as a ribosome binding site in the transcribed region, for translation. The minicircle vectors may include at least one selectable marker, for example, dihydrofolate reductase, G418, or a marker of neomycin resistance for eukaryotic cell culture; and tetracycline, kanamycin, or ampicillin resistance genes for culturing in E. coli and other prokaryotic cell culture. The minicircle producing plasmids may include at least one origin of replication to allow for the multiplication of the vector in a suitable eukaryotic or a prokaryotic host cell. Origins of replication are known in the art, as described, for example, in Genes II, Lewin, B., ed., John Wiley & Sons, New York (1985).

### 5. Compositions

The invention also provides compositions, particularly immunogenic compositions, comprising the first and second constructs described herein which may be delivered, for example, using the same or different vectors or vehicles. The first and second constructs may be administered separately, concurrently or sequentially. The immunogenic compositions may be given more than once (e.g., a “prime” administration followed by one or more “boosts”) to achieve the desired effects. The same composition can be administered in one or more priming and one or more boosting steps. Alternatively, different compositions can be used for priming and boosting.

5.1 Pharmaceutically Acceptable Components

The compositions of the present invention are suitably pharmaceutical compositions. The pharmaceutical compositions often comprise one or more “pharmaceutically acceptable carriers.” These include any carrier which does not itself induce the production of antibodies harmful to the individual receiving the composition. Suitable carriers typically are large, slowly metabolized macromolecules such as proteins, polysaccharides, polylactic acids, polyglycolic acids, polymeric amino acids, amino acid copolymers, and lipid aggregates (such as oil droplets or liposomes). Such carriers are well known to those of ordinary skill in the art. A composition may also contain a diluent, such as water, saline, glycerol, etc. Additionally, an auxiliary substance, such as a wetting or emulsifying agent, pH buffering substance, and the like, may be present. A thorough discussion of pharmaceutically acceptable components is available in Gennaro (2000) Remington: The Science and Practice of Pharmacy. 20th ed., ISBN: 0683306472.

The pharmaceutical compositions may include various salts, excipients, delivery vehicles and/or auxiliary agents as are disclosed, e.g., in U.S. patent application Publication No. 2002/0019358, published Feb. 14, 2002.

Alternatively or in addition, the pharmaceutical compositions of the present invention may include one or more transfection facilitating compounds that facilitate delivery of polynucleotides to the interior of a cell, and/or to a desired location within a cell. As used herein, the terms “transfection facilitating compound,” “transfection facilitating agent,” and “transfection facilitating material” are synonymous, and may be used interchangeably. It should be noted that certain transfection facilitating compounds may also be “adjuvants” as described infra, i.e., in addition to facilitating delivery of polynucleotides to the interior of a cell, the compound acts to alter or increase the immune response to the antigen encoded by that polynucleotide. Examples of the transfection facilitating compounds include, but are not limited to, inorganic materials such as calcium phosphate, alum (aluminium phosphate), and gold particles (e.g., “powder” type delivery vehicles); peptides that are, for example, canonic, intercell targeting (for selective delivery to certain cell types), intracell targeting (for nuclear localization or endosomal escape), and ampipathic (helix forming or pore forming); proteins that are, for example, basic (e.g., positively charged) such as histories, targeting (e.g., asialoprotein), viral (e.g., Sendai virus coat protein), and pore-forming; lipids that are, for example, cationic (e.g., DMRIE, DOSPA, DC-Chol), basic (e.g., steryl amine), neutral (e.g., cholesterol), anionic (e.g., phosphatidyl serine), and zwitterionic (e.g., DOPE, DOPC); and polymers such as dendrimers, star-polymers, “homogenous” poly-amino acids (e.g., poly-lysine, poly-arginine), “heterogeneous” poly-amino acids (e.g., mixtures of lysine & glycine), co-polymers, polyvinylpyrrolidinone (PVP), poloxamers (e.g. CRL 1005) and polyethylene glycol (PEG). A transfection facilitating material can be used alone or in combination with one or more other transfection facilitating materials. Two or more transfection facilitating materials can be combined by chemical bonding (e.g., covalent and ionic such as in lipidated polylysine, PEGylated polylysine) (Toncheva, et al., Biochim. Biophys. Acta 1380(3):354-368 (1988)), mechanical mixing (e.g., tree moving materials in liquid or solid phase such as “polylysine+cationic lipids”) (Gao and Huang, Biochemistry 35:1027-1036 (1996); Trubetskoy, et al., Biochem. Biophys. Acta 1131:311-313 (1992)), and aggregation (e.g., co-precipitation, gel forming such as in cationic lipids+poly-lactide, and polylysine+gelatin).

One category of transfection facilitating materials is cationic lipids. Examples of cationic lipids are 5-carboxyspermylglycine dioctadecylamide (DOGS) and dipalmitoyl-phophatidylethanolamine-5-carboxyspermylamide (DPPES). Cationic cholesterol derivatives are also useful, including {3β-[N—N′,N′-dimethylamino)ethane]-carbomoyl}-cholesterol (DC-Chol). Dimethyldioctdecyl-ammonium bromide (DDAB), N-(3-aminopropyl)-N,N-(bis-(2-tetradecyloxyethyl))-N-methyl-ammonium bromide (PA-DEMO), N-(3-aminopropyl)-N,N-(bis-(2-dodecyloxyethyl))-N-methyl-ammonium bromide (PA-DELO), N,N,N-tris-(2-dodecyloxy)ethyl-N-(3-amino)propyl-ammonium bromide (PA-TELO), and N1-(3-aminopropyl)((2-dodecyloxy)ethyl)-N2-(2-dodecyloxy)ethyl-1-piperazinaminium bromide (GA-LOE-BP) can also be employed in the present invention.

Non-diether cationic lipids, such as DL-1,2-doleoyl-3-dimethylaminopropyl-β-hydroxyethylammonium (DORI diester), 1-O-oleyl-2-oleoyl-3-dimethylaminopropyl-p-hydroxyethylammonium (DORI ester/ether), and their salts promote in vivo gene delivery. In some embodiments, cationic lipids comprise groups attached via a heteroatom attached to the quaternary ammonium moiety in the head group. A glycyl spacer can connect the linker to the hydroxyl group.

Specific, but non-limiting cationic lipids for use in certain embodiments of the present invention include DMRIE ((±)-N-(2-hydroxyethyl)-N,N-dimethyl-2,3-bis(tetradecyloxy)-1-propanaminium bromide), GAP-DMORIE ((±)-N-(3-aminopropyl)-N,N-dimethyl-2,3-bis(syn-9-tetradecenyloxy)-1-propanaminium bromide), and GAP-DMRIE((±)-N-(3-aminopropyl)-N,N-dimethyl-2,3-(bis-dodecyloxy)-1-propaniminium bromide).

Other specific but non-limiting cationic surfactants for use in certain embodiments of the present invention include Bn-DHRIE, DhxRIE, DhxRIE-OAc, DhxRIE-OBz and Pr-DOctRIE-OAc. These lipids are disclosed in copending U.S. patent application Ser. No. 10/725,015. In another aspect of the present invention, the cationic surfactant is Pr-DOctRIE-OAc.

Other cationic lipids include (±)-N,N-dimethyl-N-[2-(sperminecarboxamido)ethyl]-2,3-bis(dioleyloxy)-1-propaniminium pentahydrochloride (DOSPA), (±)-N-(2-aminoethyl)-N,N-dimethyl-2,3-bis(tetradecyloxy)-1-propaniminium bromide (β-aminoethyl-DMRIE or βAE-DMRIE) (Wheeler, et al., Biochim. Biophys. Acta 1280:1-11 (1996), and (±)-N-(3-aminopropyl)-N,N-dimethyl-2,3-bis(dodecyloxy)-1-propaniminium bromide (GAP-DLRIE) (Wheeler, et al., Proc. Natl. Acad. Sci. USA 93:11454-11459 (1996)), which have been developed from DMRIE.

Other examples of DMRIE-derived cationic lipids that are useful for the present invention are (±)-N-(3-aminopropyl)-N,N-dimethyl-2,3-(bis-decyloxy)-1-propanaminium bromide (GAP-DDRIE), (±)-N-(3-aminopropyl)-N,N-dimethyl-2,3-(bis-tetradecyloxy)-1-propanaminium bromide (GAP-DMRIE), (±)-N—((N″-methyl)-N′-ureyl)propyl-N,N-dimethyl-2,3-bis(tetradecyloxy-)-1-propanaminium bromide (GMU-DMRIE), (±)-N-(2-hydroxyethyl)-N,N-dimethyl-2,3-bis(dodecyloxy)-1-propanaminium bromide (DLRIE), and (±)-N-(2-hydroxyethyl)-N,N-dimethyl-2,3-bis-([Z]-9-octadecenyloxy)propyl-1-propaniminium bromide (HP-DORIE).

In the embodiments where the immunogenic composition comprises a cationic lipid, the cationic lipid may be mixed with one or more co-lipids. For purposes of definition, the term “co-lipid” refers to any hydrophobic material which may be combined with the cationic lipid component and includes amphipathic lipids, such as phospholipids, and neutral lipids, such as cholesterol. Cationic lipids and co-lipids may be mixed or combined in a number of ways to produce a variety of non-covalently bonded macroscopic structures, including, for example, liposomes, multilamellar vesicles, unilamellar vesicles, micelles, and simple films. One non-limiting class of co-lipids are the zwitterionic phospholipids, which include the phosphatidylethanolamines and the phosphatidylcholines. Examples of phosphatidylethanolamines, include DOPE, DMPE and DPyPE. In certain embodiments, the co-lipid is DPyPE which comprises two phytanoyl substituents incorporated into the diacylphosphatidylethanolamine skeleton and the cationic lipid is GAP-DMORIE, (resulting in VAXFECTIN adjuvant). In other embodiments, the co-lipid is DOPE, the CAS name is 1,2-diolyeoyl-sn-glycero-3-phosphoethanolamine.

When a composition of the present invention comprises a cationic lipid and co-lipid, the cationic lipid:co-lipid molar ratio may be from about 9:1 to about 1:9, from about 4:1 to about 1:4, from about 2:1 to about 1:2, or about 1:1.

In order to maximize homogeneity, the cationic lipid and co-lipid components may be dissolved in a solvent such as chloroform, followed by evaporation of the cationic lipid/co-lipid solution under vacuum to dryness as a film on the inner surface of a glass vessel (e.g., a Rotovap round-bottomed flask). Upon suspension in an aqueous solvent, the amphipathic lipid component molecules self-assemble into homogenous lipid vesicles. These lipid vesicles may subsequently be processed to have a selected mean diameter of uniform size prior to complexing with, for example, a codon-optimized polynucleotide of the present invention, according to methods known to those skilled in the art. For example, the sonication of a lipid solution is described in Felgner et al., Proc. Natl. Acad. Sci. USA 8: 7413-7417 (1987) and in U.S. Pat. No. 5,264,618.

In those embodiments where the composition includes a cationic lipid, polynucleotides of the present invention are complexed with lipids by mixing, for example, a plasmid in aqueous solution and a solution of cationic lipid:co-lipid as prepared herein are mixed. The concentration of each of the constituent solutions can be adjusted prior to mixing such that the desired final plasmid/cationic lipid:co-lipid ratio and the desired plasmid final concentration will be obtained upon mixing the two solutions. The cationic lipid:co-lipid mixtures are suitably prepared by hydrating a thin film of the mixed lipid materials in an appropriate volume of aqueous solvent by vortex mixing at ambient temperatures for about 1 minute. The thin films are prepared by admixing chloroform solutions of the individual components to afford a desired molar solute ratio followed by aliquoting the desired volume of the solutions into a suitable container. The solvent is removed by evaporation, first with a stream of dry, inert gas (e.g. argon) followed by high vacuum treatment.

Other hydrophobic and amphiphilic additives, such as, for example, sterols, fatty acids, gangliosides, glycolipids, lipopeptides, liposaccharides, neobees, niosomes, prostaglandins and sphingolipids, may also be included in compositions of the present invention. In such compositions, these additives may be included in an amount between about 0.1 mol % and about 99.9 mol % (relative to total lipid), about 1-50 mol %, or about 2-25 mol %.

The first and second constructs may also be encapsulated, adsorbed to, or associated with, particulate carriers. Such carriers present multiple copies-of selected constructs to the immune system. The particles can be taken up by professional antigen presenting cells such as macrophages and dendritic cells, and/or can enhance antigen presentation through other mechanisms such as stimulation of cytokine release. Examples of particulate carriers include those derived from polymethyl methacrylate polymers, as well as microparticles derived from poly(lactides) and poly(lactide-co-glycolides), known as PLG. See, e.g., Jeffery et al., 1993, Pharm. Res. 10:362-368; McGee J. P., et al., 1997, J Microencapsul. 14(2):197-210; O'Hagan D. T., et al., 1993, Vaccine 11(2):149-54.

Furthermore, other particulate systems and polymers can be used for the in vivo delivery of the compositions described herein. For example, polymers such as polylysine, polyarginine, polyornithine, spermine, spermidine, as well as conjugates of these molecules, are useful for transferring a nucleic acid of interest. Similarly, DEAE dextran-mediated transfection, calcium phosphate precipitation or precipitation using other insoluble inorganic salts, such as strontium phosphate, aluminium silicates including bentonite and kaolin, chromic oxide, magnesium silicate, talc, and the like, will find use with the present methods. See, e.g., Felgner, P. L., Advanced Drug Delivery Reviews (1990) 5:163-187, for a review of delivery systems useful for gene transfer. Peptoids (Zuckerman, R. N., et al., U.S. Pat. No. 5,831,005, issued Nov. 3, 1998) may also be used for delivery of a construct of the present invention.

Additional embodiments of the present invention are drawn to compositions comprising an auxiliary agent which is administered before, after, or concurrently with the synthetic constructs. As used herein, an “auxiliary agent” is a substance included in a composition for its ability to enhance, relative to a composition which is identical except for the inclusion of the auxiliary agent, the entry of polynucleotides into vertebrate cells in vivo, and/or the in vivo expression of polypeptides encoded by such polynucleotides. Certain auxiliary agents may, in addition to enhancing entry of polynucleotides into cells, enhance an immune response to an immunogen encoded by the polynucleotide. Auxiliary agents of the present invention include nonionic, anionic, canonic, or zwitterionic surfactants or detergents, with nonionic surfactants or detergents being preferred, chelators, DNase inhibitors, poloxamers, agents that aggregate or condense nucleic acids, emulsifying or solubilizing agents, wetting agents, gel-forming agents, and buffers.

Auxiliary agents for use in compositions of the present invention include, but are not limited to non-ionic detergents and surfactants IGEPAL CA 6300 octylphenyl-polyethylene glycol, NONIDET NP-40 nonylphenoxypolyethoxyethanol, NONIDET P-40 octylphenoxypolyethoxyethanol, TWEEN-20 polysorbate 20, TWEEN-80 polysorbate 80, PLURONIC F68 poloxamer (ave. MW: 8400; approx. MW of hydrophobe, 1800; approx. wt. % of hydrophile, 80%), PLURONIC F77 poloxamer (ave. MW: 6600; approx. MW of hydrophobe, 2100; approx. wt. % of hydrophile, 70%), PLURONIC P65 poloxamer (ave. MW: 3400; approx. MW of hydrophobe, 1800; approx. wt. % of hydrophile, 50%), TRITON X-100 4-(1,1,3,3-tetramethylbutyl)phenyl-polyethylene glycol, and TRITON X-114 (1,1,3,3-tetramethylbutyl)phenyl-polyethylene glycol; the anionic detergent sodium dodecyl sulfate (SDS); the sugar stachyose; the condensing agent DMSO; and the chelator/DNAse inhibitor EDTA, CRL 1005 (12 kpa, 5% POE), and BAK (Benzalkonium chloride 50% solution, available from Ruger Chemical Co. Inc.). In certain specific embodiments, the auxiliary agent is DMSO, NONIDET P-40 octylphenoxypolyethoxyethanol, PLURONIC F68 poloxamer (ave. MW: 8400; approx. MW of hydrophobe, 1800; approx. wt. % of hydrophile, 80%), PLURONIC F77 poloxamer (ave. MW: 6600; approx. MW of hydrophobe, 2100; approx. wt. % of hydrophile, 70%), PLURONIC P65 (ave. MW: 3400; approx. MW of hydrophobe, 1800; approx. wt. % of hydrophile, 50%), Pluronic PLURONIC L64 poloxamer (ave. MW: 2900; approx. MW of hydrophobe, 1800; approx. wt. % of hydrophile, 40%), and PLURONIC F108 poloxamer (ave. MW: 14600; approx. MW of hydrophobe, 3000; approx. wt. % of hydrophile, 80%). See, e.g., U.S. patent application Publication No. 2002/0019358, published Feb. 14, 2002.

Certain compositions of the present invention can further include one or more adjuvants before, after, or concurrently with the polynucleotide. The term “adjuvant” refers to any material having the ability to (1) alter or increase the immune response to a particular antigen or (2) increase or aid an effect of a pharmacological agent. It should be noted, with respect to polynucleotide vaccines, that an “adjuvant,” can be a transfection facilitating material. Similarly, certain “transfection facilitating materials” described supra, may also be an “adjuvant.” An adjuvant maybe used with a composition comprising a polynucleotide of the present invention. In a prime-boost regimen, as described herein, an adjuvant may be used with either the priming immunization, the booster immunization, or both. Suitable adjuvants include, but are not limited to, cytokines and growth factors; bacterial components (e.g., endotoxins, in particular superantigens, exotoxins and cell wall components); aluminium-based salts; calcium-based salts; silica; polynucleotides; toxoids; serum proteins, viruses and virally-derived materials, poisons, venoms, imidazoquiniline compounds, poloxamers, and cationic lipids.

A great variety of materials have been shown to have adjuvant activity through a variety of mechanisms. Any compound which may increase the expression, antigenicity or immunogenicity of the polypeptide is a potential adjuvant. The present invention provides an assay to screen for improved immune responses to potential adjuvants. Potential adjuvants which may be screened for their ability to enhance the immune response according to the present invention include, but are not limited to: inert carriers, such as alum, bentonite, latex, and acrylic particles; PLURONIC block polymers, such as TITERMAX (block copolymer CRL-8941, squalene (a metabolizable oil) and a microparticulate silica stabilizer); depot formers, such as Freunds adjuvant, surface active materials, such as saponin, lysolecithin, retinal, Quil A, liposomes, and PLURONIC polymer formulations; macrophage stimulators, such as bacterial lipopolysaccharide; alternate pathway complement activators, such as insulin, zymosan, endotoxin, and levamisole; and non-ionic surfactants, such as poloxamers, poly(oxyethylene)-poly(oxypropylene) tri-block copolymers. Also included as adjuvants are transfection-facilitating materials, such as those described above.

Poloxamers which may be screened for their ability to enhance the immune response according to the present invention include, but are not limited to, commercially available poloxamers such as PLURONIC surfactants, which are block copolymers of propylene oxide and ethylene oxide in which the propylene oxide block is sandwiched between two ethylene oxide blocks. Examples of PLURONIC surfactants include PLURONIC L121 poloxamer (ave. MW: 4400; approx. MW of hydrophobe, 3600; approx. wt % of hydrophile, 10%), PLURONIC L101 poloxamer (ave. MW: 3800; approx. MW of hydrophobe, 3000; approx. wt. % of hydrophile, 10%), PLURONIC L81 poloxamer (ave. MW: 2750; approx. MW of hydrophobe, 2400; approx. wt. % of hydrophile, 10%), PLURONIC L61 poloxamer (ave. MW: 2000; approx. MW of hydrophobe, 1800; approx. wt. % of hydrophile, 10%), PLURONIC L31 poloxamer (ave. MW: 1100; approx. MW of hydrophobe, 900; approx. wt. % of hydrophile, 10%), PLURONIC L122 poloxamer (ave. MW: 5000; approx. MW of hydrophobe, 3600; approx. wt. % of hydrophile, 20%), PLURONIC L92 poloxamer (ave. MW: 3650; approx. MW of hydrophobe, 2700; approx. wt. % of hydrophile, 20%), PLURONIC L72 poloxamer (ave. MW: 2750; approx. MW of hydrophobe, 2100; approx. wt. % of hydrophile, 20%), PLURONIC L62 poloxamer (ave. MW: 2500; approx. MW of hydrophobe, 1800; approx. wt. % of hydrophile, 20%), PLURONIC L42 poloxamer (ave. MW: 1630; approx. MW of hydrophobe, 1200; approx. wt. % of hydrophile, 20%), PLURONIC L63 poloxamer (ave. MW: 2650; approx. MW of hydrophobe, 1800; approx. wt. % of hydrophile, 30%), PLURONIC L43 poloxamer (ave. MW: 1850; approx. MW of hydrophobe, 1200; approx. wt. % of hydrophile, 30%), PLURONIC L64 poloxamer (ave. MW: 2900; approx. MW of hydrophobe, 1800; approx. wt. % of hydrophile, 40%), PLURONIC L44 poloxamer (ave. MW: 2200; approx. MW of hydrophobe, 1200; approx. wt. % of hydrophile, 40%), PLURONIC L35 poloxamer (ave. MW: 1900; approx. MW of hydrophobe, 900; approx. wt. % of hydrophile, 50%), PLURONIC P123 poloxamer (ave. MW: 5750; approx. MW of hydrophobe, 3600; approx. wt. % of hydrophile, 30%), PLURONIC P103 poloxamer (ave. MW: 4950; approx. MW of hydrophobe, 3000; approx. wt. % of hydrophile, 30%), PLURONIC P104 poloxamer (ave. MW: 5900; approx. MW of hydrophobe, 3000; approx. wt. % of hydrophile, 40%), PLURONIC P84 poloxamer (ave. MW: 4200; approx. MW of hydrophobe, 2400; approx. wt. % of hydrophile, 40%), PLURONIC P105 poloxamer (ave. MW: 6500; approx. MW of hydrophobe, 3000; approx. wt. % of hydrophile, 50%), PLURONIC P85 poloxamer (ave. MW: 4600; approx. MW of hydrophobe, 2400; approx. wt. % of hydrophile, 50%), PLURONIC P75 poloxamer (ave. MW: 4150; approx. MW of hydrophobe, 2100; approx. wt. % of hydrophile, 50%), PLURONIC P65 poloxamer (ave. MW: 3400; approx. MW of hydrophobe, 1800; approx. wt. % of hydrophile, 50%), PLURONIC F127 poloxamer (ave. MW: 12600; approx. MW of hydrophobe, 3600; approx. wt. % of hydrophile, 70%), PLURONIC F98 poloxamer (ave. MW: 13000; approx. MW of hydrophobe, 2700; approx. wt. % of hydrophile, 80%), PLURONIC F87 poloxamer (ave. MW: 7700; approx. MW of hydrophobe, 2400; approx. wt. % of hydrophile, 70%), PLURONIC F77 poloxamer (ave. MW: 6600; approx. MW of hydrophobe, 2100; approx. wt. % of hydrophile, 70%), PLURONIC F108 poloxamer (ave. MW: 14600; approx. MW of hydrophobe, 3000; approx. wt. % of hydrophile, 80%), PLURONIC F98 poloxamer (ave. MW: 13000; approx. MW of hydrophobe, 2700; approx. wt. % of hydrophile, 80%), PLURONIC F88 poloxamer (ave. MW: 11400; approx. MW of hydrophobe, 2400; approx. wt. % of hydrophile, 80%), PLURONIC F68 poloxamer (ave. MW: 8400; approx. MW of hydrophobe, 1800; approx. wt. % of hydrophile, 80%), PLURONIC F38 poloxamer (ave. MW: 4700; approx. MW of hydrophobe, 900; approx. wt. % of hydrophile, 80%).

Reverse poloxamers which may be screened for their ability to enhance the immune response according to the present invention include, but are not limited to PLURONIC R 31R1 reverse poloxamer (ave. MW: 3250; approx. MW of hydrophobe, 3100; approx. wt. % of hydrophile, 10%), PLURONIC R25R1 reverse poloxamer (ave. MW: 2700; approx. MW of hydrophobe, 2500; approx. wt. % of hydrophile, 10%), PLURONIC R 17R1 reverse poloxamer (ave. MW: 1900; approx. MW of hydrophobe, 1700; approx. wt. % of hydrophile, 10%), PLURONIC R 31R2 reverse poloxamer (ave. MW: 3300; approx. MW of hydrophobe, 3100; approx. wt. % of hydrophile, 20%), PLURONIC R 25R2 reverse poloxamer (ave. MW: 3100; approx. MW of hydrophobe, 2500; approx. wt. % of hydrophile, 20%), PLURONIC R 17R2 reverse poloxamer (ave. MW: 2150; approx. MW of hydrophobe, 1700; approx. wt. % of hydrophile, 20%), PLURONIC R 12R3 reverse poloxamer (ave. MW: 1800; approx. MW of hydrophobe, 1200; approx. wt. % of hydrophile, 30%), PLURONIC R 31R4 reverse poloxamer (ave. MW: 4150; approx. MW of hydrophobe, 3100; approx. wt. % of hydrophile, 40%), PLURONIC R 25R4 reverse poloxamer (ave. MW: 3600; approx. MW of hydrophobe, 2500; approx. wt. % of hydrophile, 40%), PLURONIC R 22R4 reverse poloxamer (ave. MW: 3350; approx. MW of hydrophobe, 2200; approx. wt. % of hydrophile, 40%), PLURONIC R17R4 reverse poloxamer (ave. MW: 3650; approx. MW of hydrophobe, 1700; approx. wt. % of hydrophile, 40%), PLURONIC R 25R5 reverse poloxamer (ave. MW: 4320; approx. MW of hydrophobe, 2500; approx. wt. % of hydrophile, 50%), PLURONIC R10R5 reverse poloxamer (ave. MW: 1950; approx. MW of hydrophobe, 1000; approx. wt. % of hydrophile, 50%), PLURONIC R 25R8 reverse poloxamer (ave. MW: 8550; approx. MW of hydrophobe, 2500; approx. wt. % of hydrophile, 80%), PLURONIC R 17R8 reverse poloxamer (ave. MW: 7000; approx. MW of hydrophobe, 1700; approx. wt. % of hydrophile, 80%), and PLURONIC R 10R8 reverse poloxamer (ave. MW: 4550; approx. MW of hydrophobe, 1000; approx. wt. % of hydrophile, 80%).

Other commercially available poloxamers which may be screened for their ability to enhance the immune response according to the present invention include compounds that are block copolymer of polyethylene and polypropylene glycol such as SYNPERONIC L121 (ave. MW: 4400), SYNPERONIC L122 (ave. MW: 5000), SYNPERONIC P104 (ave. MW: 5850), SYNPERONIC P105 (ave. MW: 6500), SYNPERONIC P123 (ave. MW: 5750), SYNPERONIC P85 (ave. MW: 4600) and SYNPERONIC P94 (ave. MW: 4600), in which L indicates that the surfactants are liquids, P that they are pastes, the first digit is a measure of the molecular weight of the polypropylene portion of the surfactant and the last digit of the number, multiplied by 10, gives the percent ethylene oxide content of the surfactant; and compounds that are nonylphenyl polyethylene glycol such as SYNPERONIC NP10 (nonylphenol ethoxylated surfactant-10% solution), SYNPERONIC NP30 (condensate of 1 mole of nonylphenol with 30 moles of ethylene oxide) and SYNPERONIC NP5 (condensate of 1 mole of nonylphenol with 5.5 moles of naphthalene oxide).

Other poloxamers which may be screened for their ability to enhance the immune response according to the present invention include: (a) a polyether block copolymer comprising an A-type segment and a B-type segment, wherein the A-type segment comprises a linear polymeric segment of relatively hydrophilic character, the repeating units of which contribute an average Hansch-Leo fragmental constant of about −0.4 or less and have molecular weight contributions between about 30 and about 500, wherein the B-type segment comprises a linear polymeric segment of relatively hydrophobic character, the repeating units of which contribute an average Hansch-Leo fragmental constant of about −0.4 or more and have molecular weight contributions between about 30 and about 500, wherein at least about 80% of the linkages joining the repeating units for each of the polymeric segments comprise an ether linkage; (b) a block copolymer having a polyether segment and a polycation segment, wherein the polyether segment comprises at least an A-type block, and the polycation segment comprises a plurality of cationic repeating units; and (c) a polyether-polycation copolymer comprising a polymer, a polyether segment and a polycationic segment comprising a plurality of cationic repeating units of formula —NH—R0, wherein R0 is a straight chain aliphatic group of 2 to 6 carbon atoms, which may be substituted, wherein said polyether segments comprise at least one of an A-type of B-type segment. See U.S. Pat. No. 5,656,611. Other poloxamers of interest include CRL1005 (12 kDa, 5% POE), CRL8300 (11 kDa, 5% POE), CRL2690 (12 kDa, 10% POE), CRL4505 (15 kDa, 5% POE) and CRL1415 (9 kDa, 10% POE).

Other auxiliary agents which may be screened for their ability to enhance the immune response according to the present invention include, but are not limited to, Acacia (gum arabic); the poloxyethylene ether R—O—(C2H4O)x-H (BRIJ), e.g., polyethylene glycol dodecyl ether (BRIJ 35, x=23), polyethylene glycol dodecyl ether (BRIJ 30, x=4), polyethylene glycol hexadecyl ether (BRIJ 52 x=2), polyethylene glycol hexadecyl ether (BRIJ 56, x=10), polyethylene glycol hexadecyl ether (BRIJ 58P, x=20), polyethylene glycol octadecyl ether (BRIJ 72, x=2), polyethylene glycol octadecyl ether (BRIJ 76, x=10), polyethylene glycol octadecyl ether (BRIJ® 78P, x=20), polyethylene glycol oleyl ether (BRIJ 92V, x=2), and polyoxyl 10 oleyl ether (BRIJ 97, x=10); poly-D-glucosamine (chitosan); chlorbutanol; cholesterol; diethanolamine; digitonin; dimethylsulfoxide (DMSO), ethylenediamine tetraacetic acid (EDTA); glyceryl monosterate; lanolin alcohols; mono- and di-glycerides; monoethanolamine; nonylphenol polyoxyethylene ether (NP-40); octylphenoxypolyethoxyethanol (NONIDET NP-40 from Amresco); ethyl phenol poly (ethylene glycol ether)n, n=11 (NONIDET P40 from Roche); octyl phenol ethylene oxide condensate with about 9 ethylene oxide units (NONIDET P40); IGEPAL CA 630 ((octyl phenoxy) polyethoxyethanol; structurally same as NONIDET NP-40); oleic acid; oleyl alcohol; polyethylene glycol 8000; polyoxyl 20 cetostearyl ether; polyoxyl 35 castor oil; polyoxyl 40 hydrogenated castor oil; polyoxyl 40 stearate; polyoxyethylene sorbitan monolaurate (polysorbate 20, or TWEEN-20; polyoxyethylene sorbitan monooleate (polysorbate 80, or TWEEN-80); propylene glycol diacetate; propylene glycol monostearate; protamine sulfate; proteolytic enzymes; sodium dodecyl sulfate (SDS); sodium monolaurate; sodium stearate; sorbitan derivatives (SPAN), e.g., sorbitan monopalmitate (SPAN 40), sorbitan monostearate (SPAN 60), sorbitan tristearate (SPAN 65), sorbitan monooleate (SPAN 80), and sorbitan trioleate (SPAN 85); 2,6,10,15,19,23-hexamethyl-2,6,10,14,18,22-tetracosa-hexaene (squalene); stachyose; stearic acid; sucrose; surfactin (lipopeptide antibiotic from Bacillus subtilis); dodecylpoly(ethyleneglycolether)9 (THESIT) MW 582.9; octyl phenol ethylene oxide condensate with about 9-10 ethylene oxide units (TRITON X-100); octyl phenol ethylene oxide condensate with about 7-8 ethylene oxide units (TRITON X-114); tris(2-hydroxyethyl)amine (trolamine); and emulsifying wax.

In certain adjuvant compositions, the adjuvant is a cytokine. A composition of the present invention can comprise one or more cytokines, chemokines, or compounds that induce the production of cytokines and chemokines, or a polynucleotide encoding one or more cytokines, chemokines, or compounds that induce the production of cytokines and chemokines. Examples include, but are not limited to, granulocyte macrophage colony stimulating factor (GM-CSF), granulocyte colony stimulating factor (G-CSF), macrophage colony stimulating factor (M-CSF), colony stimulating factor (CSF), erythropoietin (EPO), interleukin 2 (IL-2), interleukin-3 (IL-3), interleukin 4 (IL-4), interleukin 5 (IL-5), interleukin 6 (IL-6), interleukin 7 (IL-7), interleukin 8 (IL-8), interleukin 10 (IL-10), interleukin 12 (IL-12), interleukin 15 (IL-15), interleukin 18 (IL-18), interferon alpha (IFNα), interferon beta (IFNβ), interferon gamma (IFNγ), interferon omega (IFNΩ), interferon tau (IFNτ), interferon gamma inducing factor I (IGIF), transforming growth factor beta (TGF-β), RANTES (regulated upon activation, normal T-cell expressed and presumably secreted), macrophage inflammatory proteins (e.g., MIP-1 alpha and M3P-1 beta), Leishmania elongation initiating factor (LEIF), and Flt-3 ligand.

In certain compositions of the present invention, the polynucleotide construct may be complexed with an adjuvant composition comprising (±)-N-(3-aminopropyl)-N,N-dimethyl-2,3-bis(syn-9-tetradeceneyloxy)-1-propanaminium bromide (GAP-DMORIE). The composition may also comprise one or more co-lipids, e.g., 1,2-dioleoyl-sn-glycero-3-phosphoethanolamine (DOPE), 1,2-diphytanoyl-sn-glycero-3-phosphoethanolamine (DPyPE), and/or 1,2-dimyristoyl-glycer-3-phosphoethanolamine (DMPE). An adjuvant composition comprising GAP-DMORIE and DPyPE at a 1:1 molar ratio is referred to herein as VAXFECTIN adjuvant. See, e.g., PCT Publication No. WO 00/57917.

In other embodiments, the polynucleotide itself may function as an adjuvant as is the case when the polynucleotides of the invention are derived, in whole or in part, from bacterial DNA. Bacterial DNA containing motifs of unmethylated CpG-dinucleotides (CpG-DNA) triggers innate immune cells in vertebrates through a pattern recognition receptor (including toll receptors such as TLR 9) and thus possesses potent immunostimulatory effects on macrophages, dendritic cells and B-lymphocytes. See, e.g., Wagner, H., Curr. Opin. Microbiol. 5:62-69 (2002); Jung, J. et al., J. Immunol. 169: 2368-73 (2002); see also Klinman, D. M. et al., Proc. Natl Acad. Sci. U.S.A. 93:2879-83 (1996). Methods of using unmethylated CpG-dinucleotides as adjuvants are described in, for example, U.S. Pat. Nos. 6,207,646, 6,406,705 and 6,429,199.

The ability of an adjuvant to increase the immune response to an antigen is typically manifested by a significant increase in immune-mediated protection. For example, an increase in humoral immunity is typically manifested by a significant increase in the titre of antibodies raised to the antigen, and an increase in T-cell activity is typically manifested in increased cell proliferation, or cellular cytotoxicity, or cytokine secretion. An adjuvant may also alter an immune response, for example, by changing a primarily humoral or Th2 response into a primarily cellular, or Th1 response.

Nucleic acid molecules and/or polynucleotides of the present invention, e.g., plasmid DNA, mRNA, linear DNA or oligonucleotides, may be solubilized in any of various buffers. Suitable buffers include, for example, phosphate buffered saline (PBS), normal saline, Tris buffer, and sodium phosphate (e.g., 150 mM sodium phosphate). Insoluble polynucleotides may be solubilized in a weak acid or weak base, and then diluted to the desired volume with a buffer. The pH of the buffer may be adjusted as appropriate. In addition, a pharmaceutically acceptable additive can be used to provide an appropriate osmolarity. Such additives are within the purview of one skilled in the art. For aqueous compositions used in vivo, sterile pyrogen-free water can be used. Such formulations will contain an effective amount of a polynucleotide together with a suitable amount of an aqueous solution in order to prepare pharmaceutically acceptable compositions suitable for administration to a human.

Compositions of the present invention can be formulated according to known methods. Suitable preparation methods are described, for example, in Remington's Pharmaceutical Sciences, 16th Edition, A. Osol, ed., Mack Publishing Co., Easton, Pa. (1980), and Remington's Pharmaceutical Sciences, 19th Edition, A. R. Gennaro, ed., Mack Publishing Co., Easton, Pa. (1995). Although the composition may be administered as an aqueous solution, it can also be formulated as an emulsion, gel, solution, suspension, lyophilized form, or any other form known in the art. In addition, the composition may contain pharmaceutically acceptable additives including, for example, diluents, binders, stabilizers, and preservatives.

The following examples are included for purposes of illustration only and are not intended to limit the scope of the present invention, which is defined by the appended claims.

5.2 Dosage

The present invention is generally concerned with therapeutic compositions, i.e., to treat disease after infection. The compositions will comprise a “therapeutically effective amount” of the compositions defined herein, such that an amount of the antigen can be produced in vivo so that an immune response is generated in the individual to which it is administered. The exact amount necessary will vary depending on the subject being treated; the age and general condition of the subject to be treated; the capacity of the subject's immune system to synthesize antibodies; the degree of protection desired; the severity of the condition being treated; the particular antigen selected and its mode of administration, among other factors. An appropriate effective amount can be readily determined by one of skill in the art. Thus, a “therapeutically effective amount” will fall in a relatively broad range that can be determined through routine trials.

For example, after around 24 hours of administering the pharmaceutical compositions described herein, a dose-dependent DTH reaction occurs in human subjects receiving a dose of at least about 30 μg, 40 μg, 50 μg, 75 μg, 80 μg, 85 μg, 90 μg, 95 μg, 100 μg, 200 μg, 250 μg, 300 μg, 400 μg, 500 μg, 600 μg, 700 μg, 800 μg, 900 μg, 1000 μg, more than 1 mg, or any integer in between. Suitable, doses can be administered in more than one unit (e.g., 1 mg can be divided into two units each comprising 500 μg doses).

Dosage treatment may be a single dose schedule or a multiple dose schedule. In some embodiments, a dose of between around 30 μg to around 1 mg or above is sufficient to induce a DTH reaction to the composition. Thus, the methods of the present invention include dosages of the compositions defined herein of around 30 μg, 100 μg, 300 μg, 1 mg, or more, in order to treat HSV-2 infection.

The compositions of the present invention can be suitably formulated for injection. The composition may be prepared in unit dosage form in ampules, or in multidose containers. The polynucleotides may be present in such forms as suspensions, solutions, or emulsions in oily or preferably aqueous vehicles. Alternatively, the polynucleotide salt may be in lyophilized form for reconstitution, at the time of delivery, with a suitable vehicle, such as sterile pyrogen-free water. Both liquid as well as lyophilized forms that are to be reconstituted will comprise agents, preferably buffers, in amounts necessary to suitably adjust the pH of the injected solution. For any parenteral use, particularly if the formulation is to be administered intravenously, the total concentration of solutes should be controlled to make the preparation isotonic, hypotonic, or weakly hypertonic. Nonionic materials, such as sugars, are preferred for adjusting tonicity, and sucrose is particularly preferred. Any of these forms may further comprise suitable formulatory agents, such as starch or sugar, glycerol or saline. The compositions per unit dosage, whether liquid or solid, may contain from 0.1% to 99% of polynucleotide material.

The units dosage ampules or multidose containers, in which the polynucleotides are packaged prior to use, may comprise an hermetically sealed container enclosing an amount of polynucleotide or solution containing a polynucleotide suitable for a pharmaceutically effective dose thereof, or multiples of an effective dose. The polynucleotide is packaged as a sterile formulation, and the hermetically sealed container is designed to preserve sterility of the formulation until use.

The container in which the polynucleotide is packaged is labeled, and the label bears a notice in the form prescribed by a governmental agency, for example the U.S. Food and Drug Administration, which notice is reflective of approval by the agency under Federal law, of the manufacture, use, or sale of the polynucleotide material therein for human administration.

In most countries, federal law requires that the use of pharmaceutical agents in the therapy of humans be approved by an agency of the Federal government. Responsibility for enforcement is the responsibility of the Food and Drug Administration, which issues appropriate regulations for securing such approval, detailed in 21 U.S.C. §§301-392. Regulation for biologic material, comprising products made from the tissues of animals is provided under 42 U.S.C. §262. Similar approval is required by most foreign countries. Regulations vary from country to country, but the individual procedures are well known to those in the art.

The dosage to be administered depends to a large extent on the condition and size of the subject being treated as well as the frequency of treatment and the route of administration. Regimens for continuing therapy, including dose and frequency may be guided by the initial response and clinical judgment. The parenteral route of injection into the interstitial space of tissues is preferred, although other parenteral routes, such as inhalation of an aerosol formulation, may be required in specific administration, as for example to the mucous membranes of the nose, throat, bronchial tissues or lungs.

In preferred protocols, a formulation comprising the naked polynucleotide in an aqueous carrier is injected into tissue in amounts of from 10 μl per site to about 1 ml per site. The concentration of polynucleotide in the formulation is from about 0.1 μg/ml to about 20 mg/ml.

5.3 Routes of Administration

Once formulated, the compositions of the invention can be administered directly to the subject (e.g., as described above). Direct delivery of first and second construct-containing compositions in vivo will generally be accomplished with or without vectors, as described above, by injection using either a conventional syringe, needless devices such as BIOJECT™ or a gene gun, such as the ACCELL™ gene delivery system (PowderMed Ltd, Oxford, England) or microneedle device. The constructs can be delivered (e.g., injected) intradermally. Delivery of nucleic acid into cells of the epidermis is particularly preferred as this mode of administration provides access to skin-associated lymphoid cells and provides for a transient presence of nucleic acid (e.g., DNA) in the recipient.

Suitably, the compositions described herein are formulated for NANOPASS (Vaxxas, Brisbane, Australia) patch for microneedle administration.

In other embodiments the compositions of the invention are administered by electroporation. Such techniques greatly increases plasmid transfer across the cell plasma membrane barrier to directly or indirectly transfect plasmid into the cell cytoplasm.

Additionally, biolistic delivery systems employing particulate carriers such as gold and tungsten, are especially useful for delivering the compositions of the present invention. The particles are coated with the synthetic expression cassette(s) to be delivered and accelerated to high velocity, generally under a reduced atmosphere, using a gun powder discharge from a “gene gun.” For a description of such techniques, and apparatuses useful therefor, see, e.g., U.S. Pat. Nos. 4,945,050; 5,036,006; 5,100,792; 5,179,022; 5,371,015; and 5,478,744. In illustrative examples, gas-driven particle acceleration can be achieved with devices such as those manufactured by PowderMed Pharmaceuticals PLC (Oxford, UK) and PowderMed Vaccines Inc. (Madison, Wis.), some examples of which are described in U.S. Pat. Nos. 5,846,796; 6,010,478; 5,865,796; 5,584,807; and EP Patent No. 0500 799. This approach offers a needle-free delivery approach wherein a dry powder formulation of microscopic particles, such as polynucleotide or polypeptide particles, are accelerated to high speed within a helium gas jet generated by a hand held device, propelling the particles into a target tissue of interest. Other devices and methods that may be useful for gas-driven needle-less injection of compositions of the present invention include those provided by BIOJECT, Inc. (Portland, Oreg.), some examples of which are described in U.S. Pat. Nos. 4,790,824; 5,064,413; 5,312,335; 5,383,851; 5,399,163; 5,520,639 and 5,993,412.

Alternatively, micro-cannula- and microneedle-based devices (such as those being developed by Becton Dickinson and others) can be used to administer the compositions of the invention. Illustrative devices of this type are described in EP 1 092 444 A1, and U.S. application Ser. No. 606,909, filed Jun. 29, 2000. Standard steel cannula can also be used for intra-dermal delivery using devices and methods as described in U.S. Ser. No. 417,671, filed Oct. 14, 1999. These methods and devices include the delivery of substances through narrow gauge (about 30 G) “micro-cannula” with limited depth of penetration, as defined by the total length of the cannula or the total length of the cannula that is exposed beyond a depth-limiting feature. It is within the scope of the present invention that targeted delivery of substances including the compositions described herein can be achieved either through a single microcannula or an array of microcannula (or “microneedles”), for example 3-6 microneedles mounted on an injection device that may include or be attached to a reservoir in which the substance to be administered is contained.

In order that the invention may be readily understood and put into practical effect, particular preferred embodiments will now be described by way of the following non-limiting examples.

### EXAMPLES

### Example 1

### Construction of HSV-2 DNA Vaccine Compositions

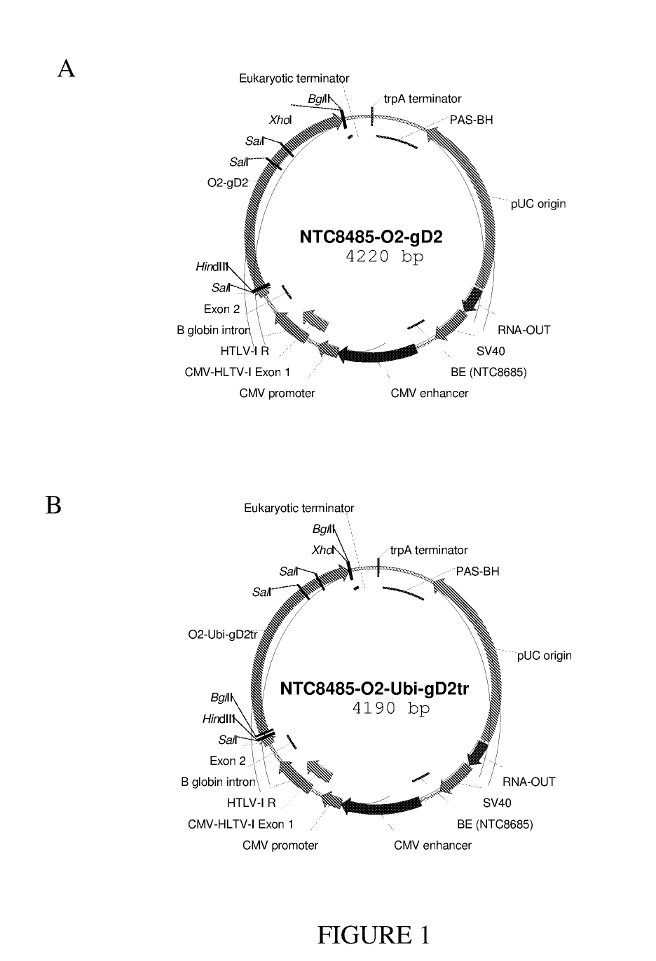

An HSV gD2 vaccine composition (COR-1) was prepared, comprising equal concentrations of the first and second constructs. The first and second synthetic coding sequences were cloned into the NTC8485 expression vector (Nature Technology Corporation (NTC), Nebraska, U.S.A.) (construct herein referred to as ‘NTC8485-O2-gD2’). The first synthetic coding sequence includes a codon optimized full length HSV-2 gD2 polynucleotide, as set forth in SEQ ID NO: 3 (see, FIG. 1A).

The second construct contains a codon optimized DNA sequence encoding a truncated form of HSV gD2 (residues 25-331) conjugated at its N-terminal end to one ubiquitin repeat (Ubi-gD2tr) (construct herein referred to as ‘NTC8485-O2-Ubi-gD2tr’). The nucleotide sequence of the second synthetic coding sequence, O2-Ubi-gD2tr, is set forth in SEQ ID NO: 2.

COR-1 is a GMP-grade 1:1 pooled mix of NTC8485-O2-gD2 and NTC8485-O2-Ubi-gD2tr, formulated with TE buffer (10 mM Tris(hydroxymethyl) amino methane hydrochloric acid (Tris-HCl), 1 mM ethylenediaminetetraacetic acid (EDTA) pH 8).

### Materials and Methods

5.4 Preparation of Constructs

The following HSV gD2 constructs were made: gD2 full length wild-type sequence (gD2), and a ubiquitinated and truncated gD2 sequence (O2-Ubi-gD2tr), as described in Nelson et al, Hum. Vaccin. Immunother, (2013), 9: 2211-5.

In brief, the O2-gD2 and O2-Ubi-gD2tr sequences were cloned into the NTC8485 vectors following the manufacturer's protocol. In brief, the ATG start codon is located in the vector immediately preceded by a SalI site. The SalI site has been demonstrated to be an effective consensus Kozak sequence for translational initiation.

The O2-gD2 and O2-Ubi-gD2tr genes are copied by PCR amplification using primers with SalI (5′ end) and BglI (3′ end) sites. Cleavage of the vectors with SalI/BglI generates sticky ends compatible with the cleaved PCR product. The insert is thus directionally and precisely cloned into the vector. The majority of recovered colonies are recombinant, since the generated sticky ends in the parental vector are not compatible.

5.5 Primer Design/Synthesis and Sequence Manipulation

Oligonucleotides for site-directed mutagenesis were designed according to the guidelines included in the relevant mutagenesis kit manuals (Quikchange II Site-directed Mutagenesis kit or Quikchange Multi Site-directed Mutagenesis Kit; Stratagene, La Jolla Calif.). These primers were synthesised and PAGE-purified by Sigma Proligo.

Oligonucleotides for whole gene synthesis were designed manually and synthesised by Sigma Proligo. The primers were supplied as standard desalted oligos. No additional purification of the oligos was carried out.

Sequence manipulation and analysis was carried out using BioEdit Version 7 (Hall, 1999) and various web-based programs including the suite of programs on Biomanager (Australian National Genome Information Service), BLAST at NCBI (http://www.ncbi.nlm.nih.gov/blast/bl2seq/wblast2.cgi), NEBcutter V2.0 from New England Biolabs (http://tools.neb.com/NEBcutter2/index.php), the Translate Tool on ExPASy (http://au.expasy.org/tools/dna.html), and the SignalP 3.0 server (http://www.cbs.dtu.dk/services/SignalP/).

5.6 Standard Molecular Biological Techniques

Restriction enzyme digests, alkaline phosphatase treatments and ligations were carried out according to the enzyme manufacturers' instructions (various manufacturers including New England Biolabs, Roche and Fermentas). Purification of DNA from agarose gels and preparation of mini-prep DNA were carried out using commercial kits (Qiagen, Bio-Rad and Macherey-Nagel).

Agarose gel electrophoresis, phenol/chloroform extraction of contaminant protein from DNA, ethanol precipitation of DNA and other basic molecular biological procedures were carried out using standard protocols, similar to those described in Current Protocols in Molecular Biology (Ebook available via Wiley InterScience; edited by Ausubel et al.).

Sequencing was carried out by the Australian Genome Research Facility (AGRF, Brisbane).

5.7 Whole Gene Synthesis

Overlapping ˜35-50mer oligonucleotides (Sigma-Proligo) were used to synthesise long DNA sequences and restriction enzyme sites incorporated to facilitate cloning. The method used to synthesise the fragments is based on that given in Smith et al. (2003). Firstly, oligos for the top or bottom strand were mixed and then phosphorylated using T4 polynucleotide kinase (PNK; New England Biolabs). The oligonucleotide mixes were purified from the PNK by a standard phenol/chloroform extraction and sodium acetate/ethanol (NaAc/EtOH) precipitation. Equal volumes of oligonucleotide mixes for the top and bottom strands were then mixed and the oligos denatured by heating at 95° C. for 2 mins. The oligos were annealed by slowly cooling the sample to 55° C. and the annealed oligos ligated using Taq ligase (New England Biolabs). The resulting fragment was purified by phenol/chloroform extraction and sodium acetate/ethanol precipitation.

The ends of the fragments were filled in and the fragments then amplified, using the outermost forward and reverse primers, with the Clontech Advantage HF 2 PCR kit (Clontech) according to the manufacturer's instructions. To fill in the ends the following PCR was used: 35 cycles of a denaturation step of 94° C. for 15 sec, a slow annealing step where the temperature was ramped down to 55° C. over 7 minutes and then kept at 55° C. for 2 min, and an elongation step of 72° C. for 6 minutes. A final elongation step for 7 min at 72° C. was then carried out. The second PCR to amplify the fragment involved: an initial denaturation step at 94° C. for 30 sec followed by 25 cycles of 94° C. for 15 sec, 55° C. 30 sec and 68° C. for 1 min, and a final elongation step of 68° C. for 3 mins.

The fragments were then purified by gel electrophoresis, digested and ligated into the relevant vector. Following transformation of E. coli with the ligation mixture, mini-preps were made for multiple colonies and the inserts sequenced. Sometimes it was not possible to isolate clones with entirely correct sequence. In those cases the errors were fixed by single or multi site-directed mutagenesis.

5.8 Site-Directed Mutagenesis

Mutagenesis was carried out using the Quikchange II Site-directed Mutagenesis kit or Quikchange Multi Site-directed Mutagenesis Kit (Stratagene, La Jolla Calif.), with appropriate PAGE (polyacrylamide gel electrophoresis)-purified primers, according to the manufacturer's instructions.

All plasmids used for vaccination were grown in the Escherichia coli strain DH5α and purified using the Nucleobond Maxi Kit (Machery-Nagal). DNA concentration was quantitated spectrophotometrically at 260 nm.

### Example 3

### Toxicity of the HSV gD2 DNA Vaccine

A clinical study was conducted to examine the safety and tolerability of intradermal injection of escalating doses of the HSV DNA vaccine (COR-1) to healthy HSV sero-negative subjects. Moreover, to determine whether COR-1 will induce anti-gD2 specific antibodies and to provide information that may lead to the prediction of an optimised dose of COR-1 to induce an efficacious immune response to protect against future HSV infection. Finally, it is the aim of the below-described experiments to determine whether the anti-gD2 antibodies are neutralizing and whether COR-1 will induce a cell mediated immune (CMI) response.

### Materials and Methods

5.9 Clinical Study Methodology

Subjects were allocated to one of the following dose groups:

4 subjects receiving 10 μg COR-1—3×10 μg injections, total exposure of 30 μg;

4 subjects receiving 30 μg COR-1—3×30 μg injections, total exposure of 90 μg;

4 subjects receiving 100 μg COR-1—3×100 μg injections, total exposure of 300 μg; and

4 subjects receiving 300 μg COR-1—3×300 μg injections, total exposure 900 μg.

4 subjects receiving 1 mg COR-1—6×500 mg (2 injections per visit), total exposure 3 mg.

The COR-1 vaccine (Batch Number: COR-1.12.N013) was administered by intradermal injection in the forearm of subjects on Day 0, Day 21 and Day 42. All subjects were HSV-1 and -2 sero-negative males, or non-pregnant non-nursing females, aged between 18 and 45 years and generally healthy. Twenty subjects were enrolled in the study and four subjects were assigned to each of the treatment groups. Two subjects withdrew from the study after the first injection, one in the 10 μg COR-1 group (withdrew consent) and one in the 1 mg group (withdrew due to inability to comply with the protocol). Two replacement subjects were then enrolled and assigned to these groups (Total n=22). A total of 20 subjects completed the study as planned, i.e., four from each treatment group. No subjects were withdrawn from the study due to adverse effects (AE).

All AE and serious AE (SAE) were assessed according to the FDA Guidance for Industry (2007): Toxicity Grading Scale for Healthy Adults and Adolescent Volunteers Enrolled in Preventative Vaccine Clinical Trials.

### Example 3

Serum samples collected within 60 minutes prior to each vaccination on days 0, 21 and 42 and at the final study visit (day 63) were analysed for the presence of anti-HSV gD2 antibodies and neutralizing anti-HSV gD2 antibodies.

Induration was frequently reported, occurring in at least one subject in each treatment group at some point during the vaccination phase.

The incidence of induration tended to be greater in the 1 mg COR-1 treatment group compared to the lower dose treatment groups. In this group, induration was observed from 45 minutes until two days after each vaccination. The occurrence of induration in the other treatment groups tended to be more sporadic and was not reported at all time points after each vaccination. In all treatment groups, any induration reported had resolved by the next visit three weeks later.

All injection site reactions were classified as mild in intensity.

### Example 4

### HSV Cell-Mediated Immunity

T-cell responses to 11 groups of overlapping HSV-specific peptides were assessed by measuring IFN-γ production in peripheral blood mononuclear cells (PBMC) using Enzyme Linked ImmunoSPOT (ELISPOT) assay. There were no dose-related trends observed in the ELISPOT results.

IFN-γ production was induced in PBMC from 19 of the 20 subjects who completed the study as planned. Accordingly, a clear and substantial cellular immune response to the COR-1 vaccine was observed. The response rates were similar in all treatment groups with 100% of subjects responding to the COR-1 vaccine in the 10 μg, 30 μg, 300 μg and 1 mg groups, and 75% responding in the 100 μg group.

For the Intent To Treat (ITT) population, one subject in the 10 μg group and one subject in the 1 mg group did not respond to the COR-1 vaccine. These subjects withdrew from the study prematurely and received only one vaccination.

No cellular response was observed in the negative control testing unspecific DNA reactivity (data not provided), providing supporting evidence that the cellular response observed is specific for HSV gD2.

### Materials and Methods

### HSV gD2 IFN-γ ELISPOT

ELISPOT plates were coated with capture antibody. This involved diluting the capture mAb (1-DK) to 5 μg/mL in freshly prepared and filtered 0.1 M NaHCO₃ (pH8.2-8.6), adding 75 μL of the diluted capture Ab to each well and then incubating the plates (covered in foil) overnight at 4° C. The plates were washed with 200 μL complete Roswell Park Memorial Institute medium (cRPML)/well. 200 μL/well of 10% FCS in cRPML (filtered with a 0.2 μm filter) were then added and the plates incubated (covered in foil) for 2 hours at room temperature.

While the plates were being blocked, the human PBMC were prepared. PBMC were thawed at 37° C. water-bath and transferred into 50 mL tubes. 10 mL of pre-warmed cRPMI with 10% FCS was added to the thawed PBMC and spun at 1200 rpm for 5 mins. The PBMC were washed in 10 mL pre-warmed cRPMI before spinning at 1200 rpm for 5 mins. The supernatant was discarded and the pellet resuspended in 2 mL 10% FCS cRPML. A sample of the cells was stained with trypan blue and counted on a haemocytometer. Cell suspensions were adjusted to a concentration of 1×10⁶ cells/mL.

The blocking solution was removed from the plates and the wells washed with cRPMI. 20 μL of IL-12 (1 μg/mL in cRPMI) were added per mL of PMBC. 200 μg of peptide was added per mL of PBMC. 100 μL of PBMC (1×10⁵/100 μL) and 100 μL of peptidesolution were added to each well. Pooled overlapping gD2 peptides were used (synthesized by Mimotope). The plates were covered with foil and incubated overnight at 37° C. in a 5% CO₂ incubator. Up until this point the experiment was performed under sterile conditions, from this point on it was no longer necessary.

The plates were washed six times with PBS-T (0.02% Tween-20 in PBS). The biotinylated detection mAb (7-B6-1) was diluted to 1 μg/mL in PBS-T containing 0.5% FCS. 75 μL were added to each well and the plates (covered with foil) incubated for 2-4 h at RT. The plates were then washed six times with PBS-T. Strepavidin-HRP (1 mg/mL stock) was diluted 1:400 in PBS-T containing 0.5% FCS and 75 μL added per well. The plates were incubated (covered in foil) for 1 h at room temperature. The plates were washed three times with PBS-T then three times with PBS only.

DAB substrate solution (Sigma) was prepared as per the manufacturer's instructions. 75 μL of substrate was added to each well. Plates were washed in tap water six times to stop colour development. The back cover was removed to allow the bottom side of the wells to be rinsed. The plates were left to dry overnight and stored in the dark.

### Example 5

### Delayed Type Hypersensitivity Response

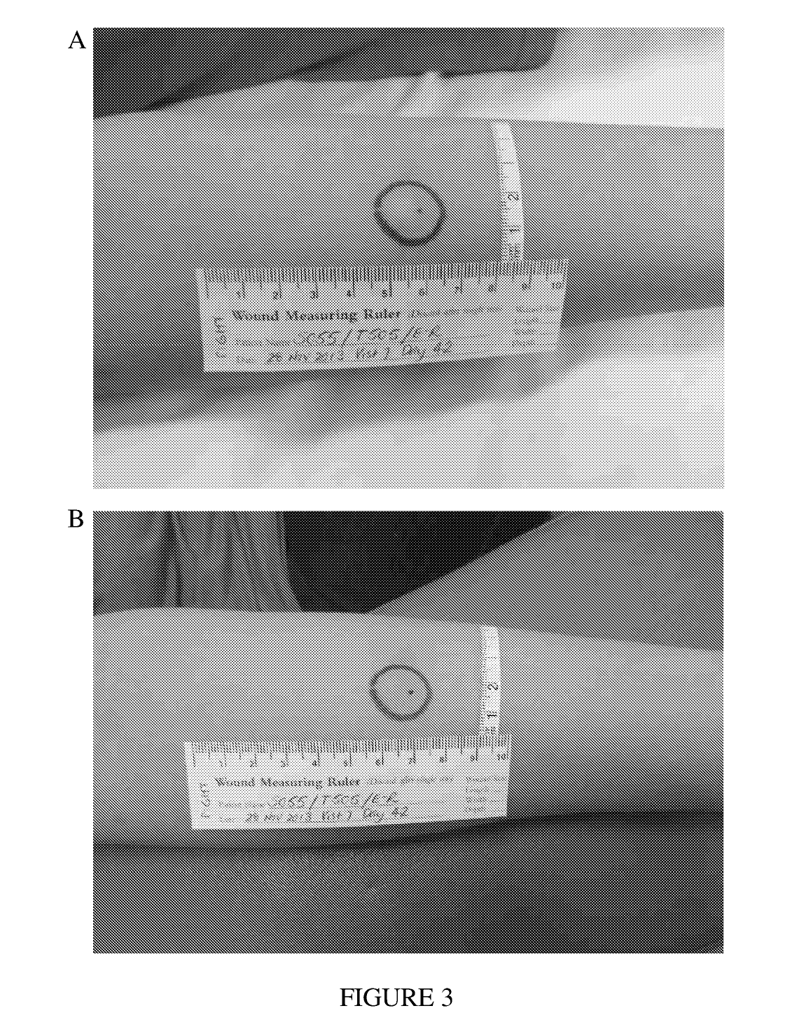

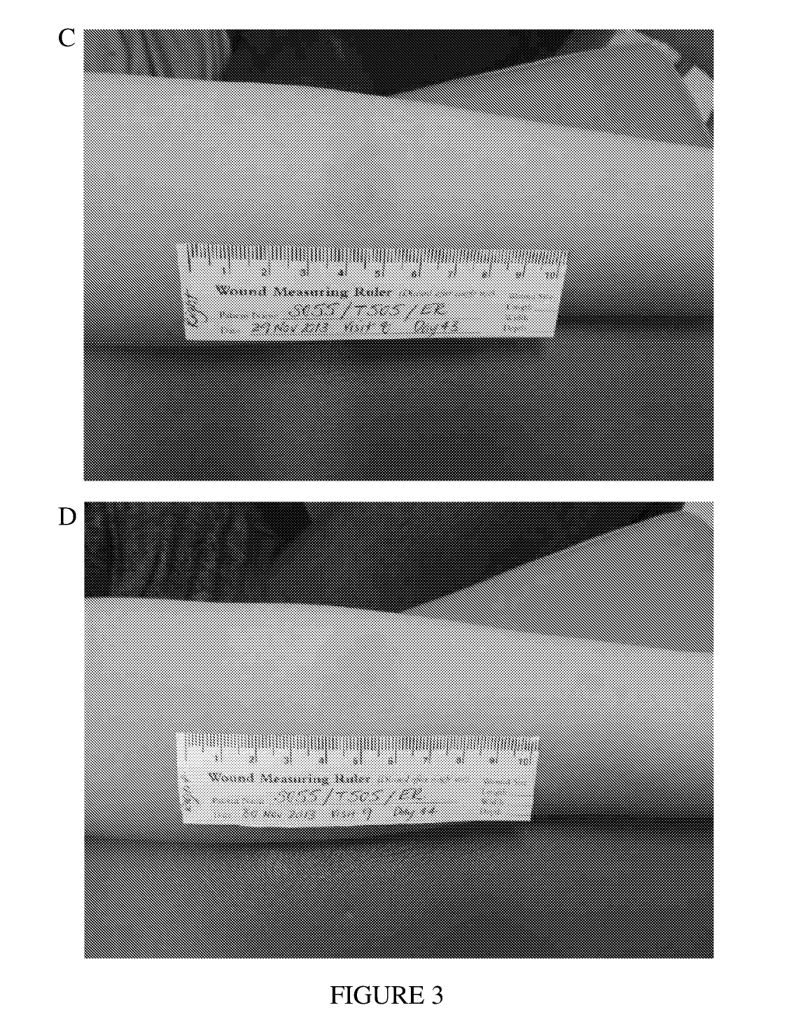

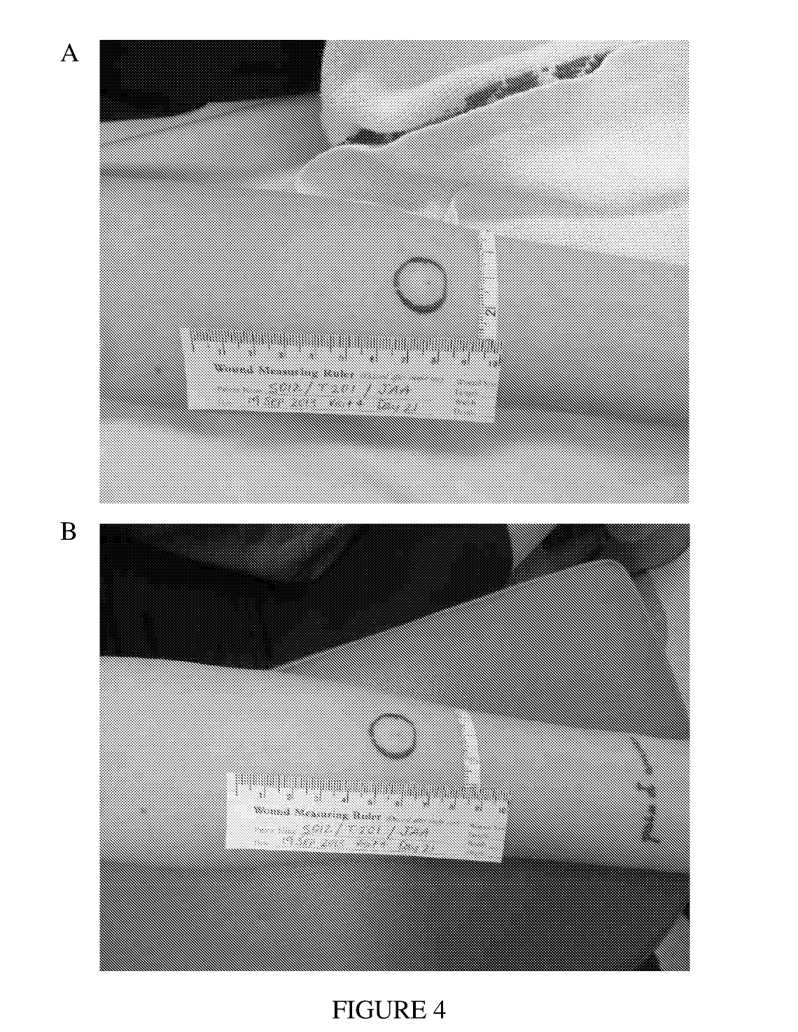

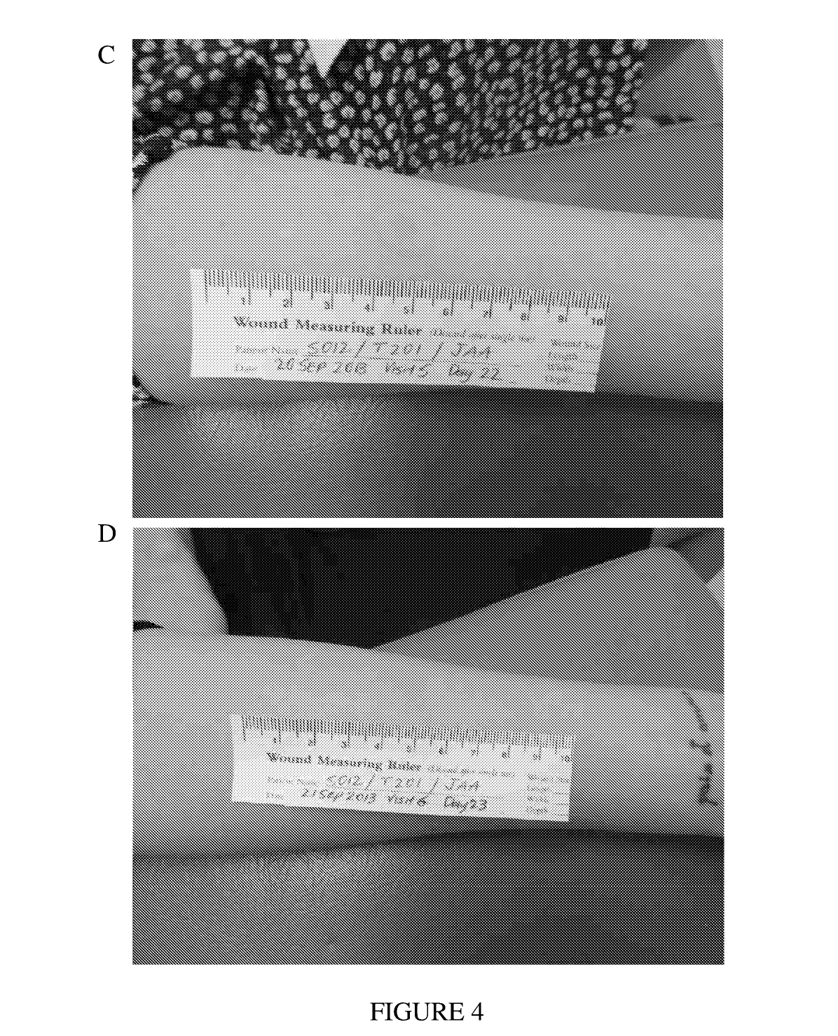

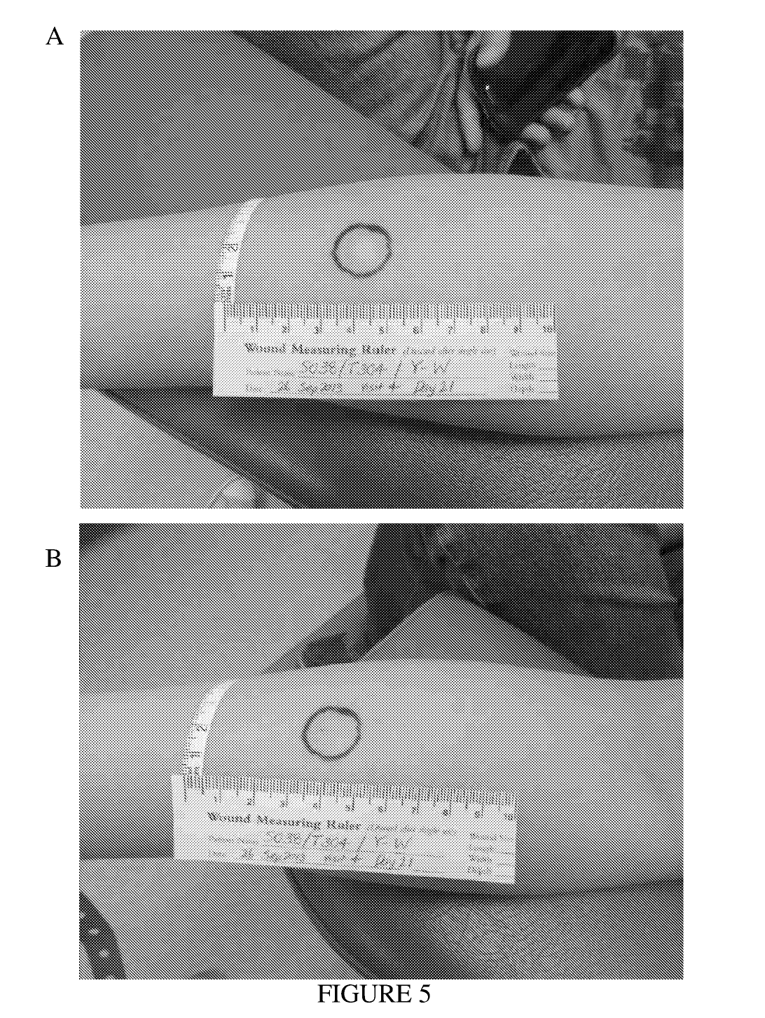

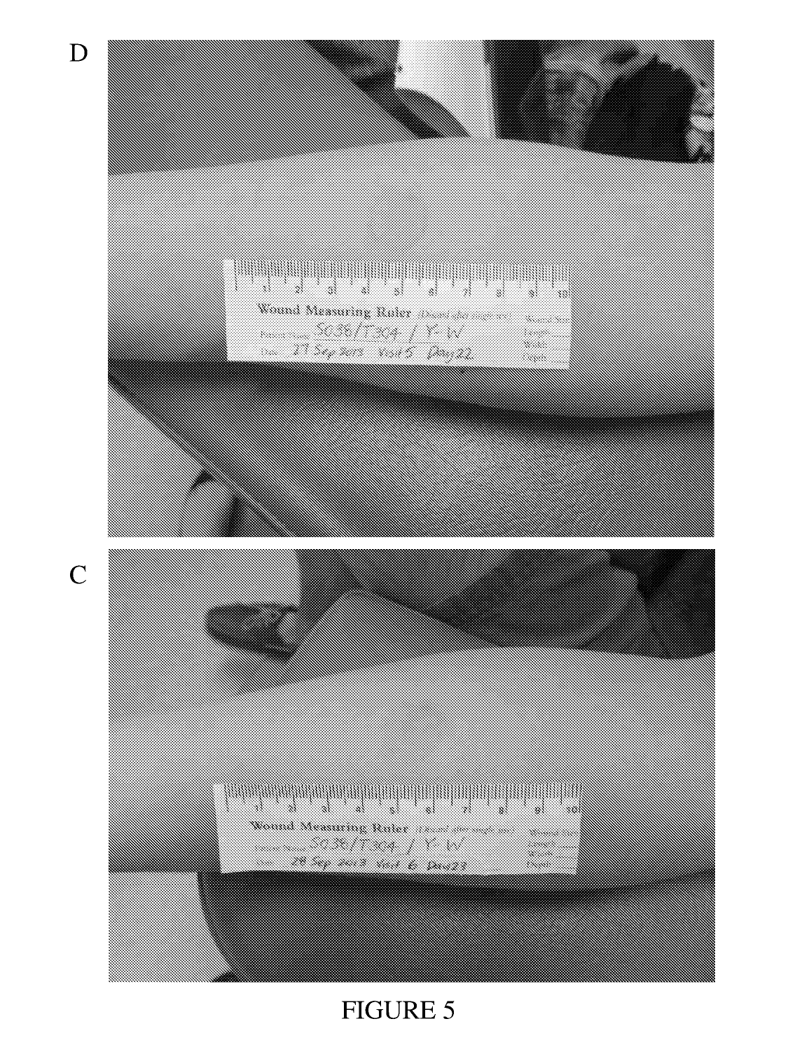

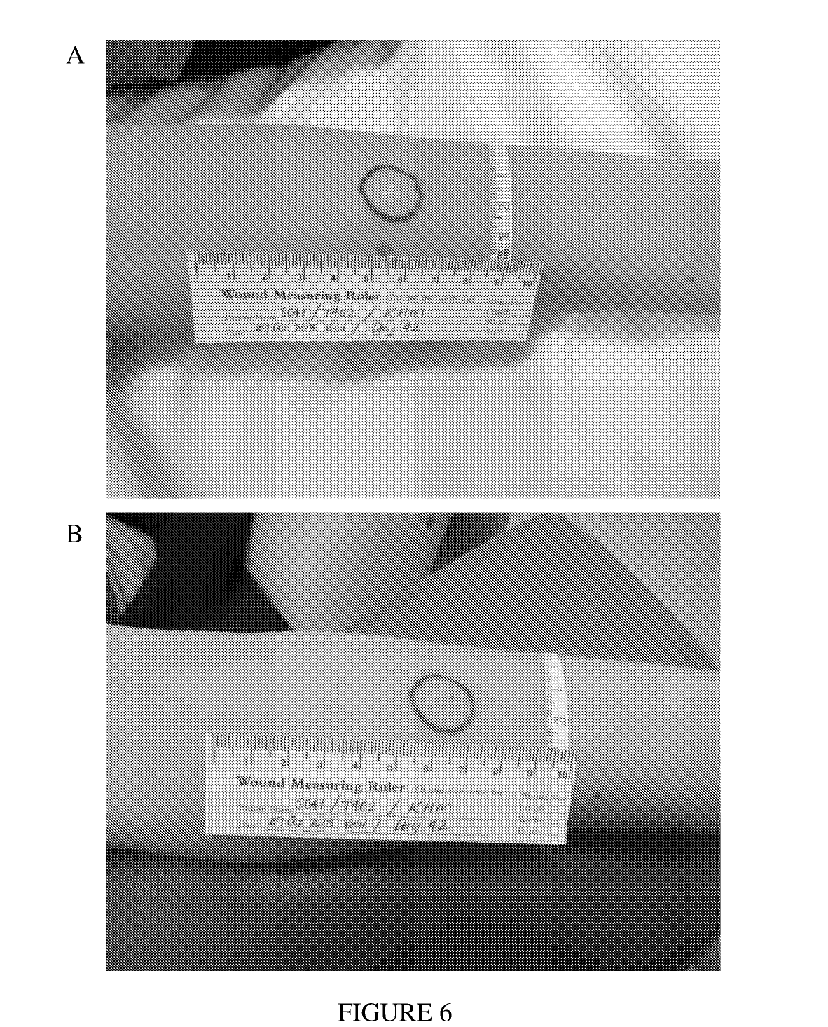

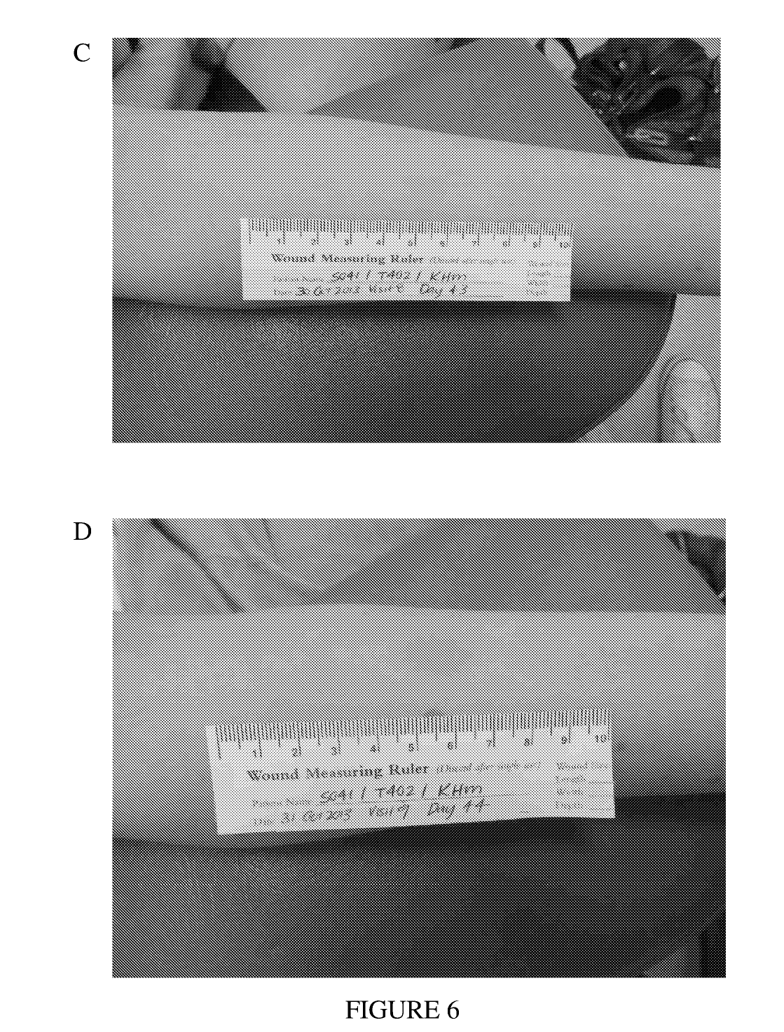

For each vaccine dosage, the intradermal injection sites were photographed immediately, 45 minutes, 24 hours and 48 hours after each injection (see, FIGS. 2-6). These photographs were used to assess injection site reactions.

An analysis on the size of the erythema observed from the photographs taken at one and two days after each vaccination. It should be noted that whilst the photographs were not taken for this specific purpose, a paper ruler was included in all but 3 photographs. The results of this analysis are detailed in Table 5.

The post hoc analysis revealed that at the higher doses of the vaccine, the size of the erythema increased on day two compared to the size of the erythema observed at day one. The incidence of erythema tended to be greater in the 100 μg, 300 μg and 1 mg COR-1 treatment groups compared to the lower dose treatment groups. In these groups, erythema was observed from 45 minutes until two days after each vaccination. The occurrence of erythema in the 10 μg and 30 μg COR-1 groups tended to be more sporadic and was not reported at all time points after each vaccination. There was no measurable erythema in the 10 μg dose treatment group. In all treatment groups, any erythema reported had resolved by the next visit three weeks later.

This result is indicative of a delayed type hypersensitivity (DTH) reaction, which is a cell mediated immune response and not an antibody-mediated response. This is not, therefore, inconsistent with the lack of observation of an antibody response.

The analysis of the photographs also revealed a dose response with the size of the erythema observed increasing with increasing doses of the vaccine.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

TABLE 5

| | | | | | | | | | | 1 mg Injection | | 1 mg Injection | |
|---|---|---|---|---|---|---|---|---|---|---|---|---|---|
| | | 10 mcg | | 30 mcg | | 100 mcg | | 300 mcg | | Site 1 | | Site 2 | |
| Scheduled Time point | Result | (N = 5) | | (N = 4) | | (N = 4) | | (N = 4) | | (N = 5) | | (N = 5) | |
| Baseline | No Symptoms | 5 | (100%) | 4 | (100%) | 4 | (100%) | 4 | (100%) | 5 | (100%) | 5 | (100%) |
| Day 0 (45 min post-dose) | No Symptoms | 5 | (100%) | 4 | (100%) | 1 | (25%) | 2 | (50%) | 1 | (20%) | 0 | (0%) |
| | NM | 0 | (0%) | 0 | (0%) | 3 | (75%) | 2 | (50%) | 4 | (80%) | 5 | (100%) |
| Day 1 | No Symptoms | 5 | (100%) | 3 | (75%) | 1 | (25%) | 0 | (0%) | 0 | (0%) | 0 | (0%) |
| | NM | 0 | (0%) | 0 | (0%) | 1 | (25%) | 0 | (0%) | 1 | (20%) | 0 | (0%) |
| | ≦0.5 cm erythema | 0 | (0%) | 1 | (25%) | 2 | (50%) | 1 | (25%) | 1 | (20%) | 3 | (60%) |
| | 1.0 cm erythema | 0 | (0%) | 0 | (0%) | 0 | (0%) | 3 | (75%) | 3 | (60%) | 2 | (40%) |
| | 1.5 cm erythema | 0 | (0%) | 0 | (0%) | 0 | (0%) | 0 | (0%) | 0 | (0%) | 0 | (0%) |
| | ≧2.0 cm erythema | 0 | (0%) | 0 | (0%) | 0 | (0%) | 0 | (0%) | 0 | (0%) | 0 | (0%) |
| Day 2 | No Symptoms | 5 | (100%) | 2 | (50%) | 1 | (25%) | 0 | (0%) | 0 | (0%) | 0 | (0%) |
| | NM | 0 | (0%) | 0 | (0%) | 0 | (0%) | 0 | (0%) | 0 | (0%) | 0 | (0%) |
| | ≦0.5 cm erythema | 0 | (0%) | 1 | (25%) | 1 | (25%) | 0 | (0%) | 1 | (20%) | 0 | (0%) |
| | 1.0 cm erythema | 0 | (0%) | 1 | (25%) | 2 | (50%) | 1 | (25%) | 0 | (0%) | 4 | (80%) |
| | 1.5 cm erythema | 0 | (0%) | 0 | (0%) | 0 | (0%) | 3 | (75%) | 0 | (0%) | 0 | (0%) |
| | ≧2.0 cm erythema | 0 | (0%) | 0 | (0%) | 0 | (0%) | 0 | (0%) | 4 | (80%) | 1 | (20%) |
| Day 21 (Pre-dose) | No Symptoms | 4 | (80%) | 4 | (100%) | 4 | (100%) | 4 | (100%) | 4 | (80%) | 4 | (80%) |
| Day 21 (45 min post-dose) | No Symptoms | 2 | (40%) | 2 | (50%) | 0 | (0%) | 2 | (50%) | 0 | (0%) | 0 | (0%) |
| | NM | 2 | (40%) | 2 | (50%) | 4 | (100%) | 2 | (50%) | 4 | (80%) | 4 | (80%) |
| Day 22 | No Symptoms | 4 | (80%) | 0 | (0%) | 0 | (0%) | 1 | (25%) | 1 | (20%) | 0 | (0%) |
| | NM | 0 | (0%) | 0 | (0%) | 0 | (0%) | 2 | (50%) | 0 | (0%) | 0 | (0%) |
| | ≦0.5 cm erythema | 0 | (0%) | 4 | (100%) | 3 | (75%) | 0 | (0%) | 0 | (0%) | 3 | (60%) |
| | 1.0 cm erythema | 0 | (0%) | 0 | (0%) | 1 | (25%) | 1 | (25%) | 2 | (40%) | 1 | (20%) |
| | 1.5 cm erythema | 0 | (0%) | 0 | (0%) | 0 | (0%) | 0 | (0%) | 1 | (20%) | 0 | (0%) |
| | ≧2.0 cm erythema | 0 | (0%) | 0 | (0%) | 0 | (0%) | 0 | (0%) | 0 | (0%) | 0 | (0%) |
| Day 23 | No Symptoms | 4 | (80%) | 0 | (0%) | 0 | (0%) | 0 | (0%) | 0 | (0%) | 0 | (0%) |
| | NM | 0 | (0%) | 0 | (0%) | 0 | (0%) | 1 | (25%) | 0 | (0%) | 0 | (0%) |
| | ≦0.5 cm erythema | 0 | (0%) | 3 | (75%) | 3 | (75%) | 0 | (0%) | 2 | (40%) | 1 | (20%) |
| | 1.0 cm erythema | 0 | (0%) | 1 | (25%) | 0 | (0%) | 1 | (25%) | 0 | (0%) | 2 | (40%) |
| | 1.5 cm erythema | 0 | (0%) | 0 | (0%) | 1 | (25%) | 2 | (50%) | 1 | (20%) | 1 | (20%) |
| | ≧2.0 cm erythema | 0 | (0%) | 0 | (0%) | 0 | (0%) | 0 | (0%) | 1 | (20%) | 0 | (0%) |
| Day 42 (Pre-dose) | No Symptoms | 4 | (80%) | 4 | (100%) | 4 | (100%) | 4 | (100%) | 4 | (80%) | 4 | (80%) |
| Day 42 (45 min post-dose) | No Symptoms | 2 | (40%) | 2 | (50%) | 0 | (0%) | 0 | (0%) | 0 | (0%) | 0 | (0%) |
| | NM | 2 | (40%) | 2 | (50%) | 4 | (100%) | 4 | (100%) | 4 | (80%) | 4 | (80%) |
| Day 43 | No Symptoms | 3 | (60%) | 2 | (50%) | 1 | (25%) | 0 | (0%) | 0 | (0%) | 1 | (20%) |
| | NM | 1 | (20%) | 0 | (0%) | 0 | (0%) | 0 | (0%) | 1 | (20%) | 0 | (0%) |
| | ≦0.5 cm erythema | 0 | (0%) | 2 | (50%) | 1 | (25%) | 3 | (75%) | 2 | (40%) | 2 | (40%) |
| | 1.0 cm erythema | 0 | (0%) | 0 | (0%) | 2 | (50%) | 1 | (25%) | 0 | (0%) | 0 | (0%) |
| | 1.5 cm erythema | 0 | (0%) | 0 | (0%) | 0 | (0%) | 0 | (0%) | 1 | (20%) | 1 | (20%) |
| | ≧2.0 cm erythema | 0 | (0%) | 0 | (0%) | 0 | (0%) | 0 | (0%) | 0 | (0%) | 0 | (0%) |
| Day 44 | No Symptoms | 3 | (60%) | 0 | (0%) | 1 | (25%) | 0 | (0%) | 0 | (0%) | 0 | (0%) |
| | NM | 1 | (20%) | 0 | (0%) | 0 | (0%) | 0 | (0%) | 0 | (0%) | 0 | (0%) |
| | ≦0.5 cm erythema | 0 | (0%) | 3 | (75%) | 1 | (25%) | 1 | (25%) | 1 | (20%) | 0 | (0%) |
| | 1.0 cm erythema | 0 | (0%) | 1 | (25%) | 2 | (50%) | 1 | (25%) | 1 | (20%) | 2 | (40%) |
| | 1.5 cm erythema | 0 | (0%) | 0 | (0%) | 0 | (0%) | 2 | (50%) | 1 | (20%) | 1 | (20%) |
| | ≧2.0 cm erythema | 0 | (0%) | 0 | (0%) | 0 | (0%) | 0 | (0%) | 1 | (20%) | 1 | (20%) |
| Day 63/Early Termination | No Symptoms | 5 | (100%) | 4 | (100%) | 4 | (100%) | 4 | (100%) | 5 | (100%) | 5 | (100%) |

NM = Not measurable from photo.

### BIBLIOGRAPHY

Agrawal, S., Importance of nucleotide sequence and chemical modifications of antisense oligonucleotides, Biochim Biophys Acta, (1999), 1489: 53-68.
Berge, S. M., Bighley L. D., Monkhouse D. C., Pharmaceutical Salts, J. Pharm. Sci., (1977), 66: 1-19.
Darquet, A.-M., Cameron, B., Wils, P., Scherman, D., Crouzet, J., A new DNA vehicle for nonviral gene delivery: supercoiled minicircle. Gene Ther., (1997), 4: 1341-1349.
Food and Drug Administration, Points to consider on plasmid DNA vaccines for preventive infectious disease indications. Federal Register, (1996), 61 FR 68269-68270.
Food and Drug Administration, Guidance for industry: considerations for plasmid DNA vaccines for infectious disease indications. Federal Register, (2007), 72 FR 61172.
Geary, R. S., Watanabe, T. A., Truong, L., Pharmacokinetic properties of 2′-O-(2-methoxyethyl)-modified oligonucleotide analogs in rats. J. Pharmacol. Exp. Ther., (2001), 296: 890-897.
Gupta, R., Warren, T., Wald, A., Genital herpes. Lancet, (2007), 370: 2127-2137.
Kopp, S. J., Storti, C. S., Muller, W. J., Herpes simplex virus-2 glycoprotein interaction with HVEM influences virus-specific recall cellular responses at the mucosa, Clin. Dev. Immunol., (2012), 284104: 1-10.
Looker, K. J., Garnett, G. P., Schmid, G. P., An estimate of the global prevalence and incidence of herpes simplex virus type 2 infection. Bull. WHO, (2008), 86, 805-812.
Luke, J., Carnes, A. E., Hodgson, C. P., Williams, J. A., Improved antibiotic-free DNA vaccine vectors utilizing a novel RNA based plasmid selection system. Vaccine, (2009), 27: 6454-6459.
Luke J., Vincent, J. M., Du, S. X., Whalen, B., Leen, A., Hodgson, C. P., Williams, J. A., Improved antibiotic-free plasmid vector design by incorporation of transient expression enhances. Gene Ther., (2011), 18: 334-343.
Nelson J., Rodriguez, S., Finlayson, N., Williams, J., Carnes, A., Antibiotic-free production of a herpes simplex virus 2 DNA vaccine in a high yield cGMP process. Hum. Vaccin. Immunother., (2013), 9(10): 2211-5.
Peng, T., Zhu, J., Klock, A., Phasouk, K., Huang, M. L., Koelle, D. M., Wald, A., Corey, L., Evasion of the mucosal innate immune system by herpes simplex virus type 2. J. Virol., (2009), 83: 12559-12568.
Perretti, S., Shaw, A., Blanchard, J., Bohm, R., Morrow, G., Lifson, J. D., Gettie, A., Pope, M., Immunomodulatory effects of HSV-2 infection on immature macaque dendritic cells modify innate and adaptive responses. Blood, (2005), 106: 1305-1313.
Pichler A. J., Yerly D., Delayed drug hypersensitivity: models of T-cell stimulation. Br J Clin Pharmacol., (2011), 71: 701-707.
Spear, P. G., Eisenberg, R. J., Cohen, G. H., Three classes of cell surface receptors for alphaherpesvirus entry. Virology, (2000), 275: 1-8.
Stefanidou, M., Ramos, I., Casullo, V. M., Trepanier, J. B., Rosenbaum, S., Fernandez-Sesma, A., Herold, B. C., Herpes simplex virus 2 (HSV-2) prevents dendritic cell maturation, induces apoptosis and triggers release of proinflammatory cytokines: Potential links to HSV-HIV synergy. J. Virol., (2013), 87: 1443-1453.
Tilton, J. C., Manion, M. M., Luskin, M. R., Johnson, A. J., Patamawenu, A. A., Hallahan, C. W., Cogliano-Shutta, N. A., Mican, J. M., Davey, R. T., Kottilil, S., Lifson, J. D., Metcalf, J. A., Lempicki, R. A., Connors, M., Human Immunodeficiency Virus Viremia Induces Pasmacytoid Dendritic Cell Activation In Vivo and Diminished Alpha Interferon Production In Vitro. Am. Soc. Microbiol., (2008), 82(8): 3997-4006.
Williams, J. A., Carnes, A. E., Hodgson, C. P., Plasmid DNA Vaccine vector design: impact on efficacy, safety and upstream production. Biotechnol. Adv., (2009), 27(4): 353-370.
Williams, J. A., Vector design for improved DNA vaccine efficacy, Safety and Production. Vaccines, (2013), 1: 225-249.

What is claimed is:

1. A method of treating a herpes simplex virus (HSV) infection in a subject, the method comprising administering concurrently to the subject an effective amount of a construct system that comprises a first construct and a second construct, wherein the first construct comprises a first synthetic coding sequence that is distinguished from a wild-type HSV gD2 coding sequence by the replacement of selected codons in the wild-type HSV gD2 coding sequence with synonymous codons that have a higher immune response than the selected codons, wherein the codon replacements are selected from TABLE 1, wherein at least 70% of the codons of the first synthetic coding sequence are synonymous codons according to TABLE 1, and wherein the first synthetic coding sequence is operably connected to a regulatory nucleic acid sequence and wherein the second construct comprises a second synthetic coding sequence that is distinguished from a wild-type HSV gD2 coding sequence by replacement of selected codons in the wild-type HSV gD2 coding sequence with synonymous codons that have a higher immune response than the selected codons, wherein the codon replacements are selected from TABLE 1, wherein at least 70% of the codons of the second synthetic coding sequence are synonymous codons according to TABLE 1, and wherein the second synthetic coding sequence is operably connected to a regulatory nucleic acid sequence and to a nucleic acid sequence that encodes a protein-destabilizing element that increases processing and presentation of the polypeptide through the class I major histocompatibility (MHC) pathway, wherein TABLE 1 is as follows:
TABLE 1

| First | Synonymous | First | Synonymous | First | Synonymous |
|---|---|---|---|---|---|
| Codon | Codon | Codon | Codon | Codon | Codon |
| Ala^{GCG} | Ala^{GCT} | Ile^{ATA} | Ile^{ATC} | Ser^{AGT} | Ser^{TCG} |
| Ala^{GCG} | Ala^{GCC} | Ile^{ATA} | Ile^{ATT} | Ser^{AGT} | Ser^{TCT} |
| Ala^{GCA} | Ala^{GCT} | Ile^{ATT} | Ile^{ATC} | Ser^{AGT} | Ser^{TCA} |
| Ala^{GCA} | Ala^{GCC} | | | Ser^{AGT} | Ser^{TCC} |
| Ala^{GCC} | Ala^{GCT} | Leu^{TTA} | Leu^{CTG} | Ser^{AGC} | Ser^{TCG} |
| | | Leu^{TTA} | Leu^{CTC} | Ser^{AGC} | Ser^{TCT} |
| Arg^{CGG} | Arg^{CGA} | Leu^{TTA} | Leu^{CTA} | Ser^{AGC} | Ser^{TCA} |
| Arg^{CGG} | Arg^{CGC} | Leu^{TTA} | Leu^{CTT} | Ser^{AGC} | Ser^{TCC} |
| Arg^{CGG} | Arg^{CGT} | Leu^{TTA} | Leu^{TTG} | Ser^{TCC} | Ser^{TCG} |
| Arg^{CGG} | Arg^{AGA} | Leu^{TTG} | Leu^{CTG} | Ser^{TCA} | Ser^{TCG} |
| Arg^{AGG} | Arg^{CGA} | Leu^{TTG} | Leu^{CTC} | Ser^{TCT} | Ser^{TCG} |
| Arg^{AGG} | Arg^{CGC} | Leu^{TTG} | Leu^{CTA} | | |
| Arg^{AGG} | Arg^{CGT} | Leu^{TTG} | Leu^{CTT} | Thr^{ACT} | Thr^{ACG} |
| Arg^{AGG} | Arg^{AGA} | Leu^{CTT} | Leu^{CTG} | Thr^{ACT} | Thr^{ACC} |
| | | Leu^{CTT} | Leu^{CTC} | Thr^{ACT} | Thr^{ACA} |
| Asn^{AAT} | Asn^{AAC} | Leu^{CTA} | Leu^{CTG} | Thr^{ACA} | Thr^{ACG} |
| | | Leu^{CTA} | Leu^{CTC} | Thr^{ACA} | Thr^{ACC} |
| Asp^{GAT} | Asp^{GAC} | | | Thr^{ACC} | Thr^{ACG} |
| | | Phe^{TTC} | Phe^{TTT} | | |
| Cys^{TGT} | Cys^{TGC} | | | Tyr^{TAT} | Tyr^{TAC} |
| | | Pro^{CCG} | Pro^{CCC} | | |
| Glu^{GAG} | Glu^{GAA} | Pro^{CCG} | Pro^{CCT} | Val^{GTA} | Val^{GTG} |
| | | Pro^{CCA} | Pro^{CCC} | Val^{GTA} | Val^{GTC} |
| Gly^{GGC} | Gly^{GGA} | Pro^{CCA} | Pro^{CCT} | Val^{GTA} | Val^{GTT} |
| Gly^{GGT} | Gly^{GGA} | Pro^{CCT} | Pro^{CCC} | Val^{GTT} | Val^{GTG} |
| Gly^{GGG} | Gly^{GGA} | | | Val^{GTT} | Val^{GTC} |

2. The method according to claim 1, further comprising identifying that the subject has an HSV-2 infection prior to administering concurrently the first and second constructs.

3. The method according to claim 1 or claim 2, wherein the protein-destabilizing element is selected from the group consisting of a destabilizing amino acid at the amino-terminus of the polypeptide, a PEST sequence and a ubiquitin molecule.

4. The method according to any one of claims 1 to 3, wherein the protein-destabilizing element is a ubiquitin molecule.

5. The method according to any one of claims 1 to 5, wherein the first synthetic coding sequence comprises the polynucleotide sequence set forth in SEQ ID NO: 3.

6. The method according to any one of claims 1 to 5, wherein the second synthetic coding sequence comprises the polynucleotide sequence set forth in SEQ ID NO: 4.

7. The method according to any one of claims 1 to 6, wherein the first construct and the second construct are contained in one or more expression vectors.

8. The method according to claim 7, wherein the expression vector is free of a signal or targeting sequence.

9. The method according to claim 7 or claim 8, wherein the expression vector does not include an antibiotic-resistance marker.

10. The method according to any one of claims 7 to 9, wherein the expression vector is NTC8485 or NTC8685.

11. The method according to any one of claims 1 to 10, wherein at least 75% of codons in the first synthetic coding sequence and the second synthetic coding sequence are synonymous codons selected from TABLE 1.

12. The method according to any one of claims 1 to 11, wherein at least 80% of codons in the first synthetic coding sequence and the second synthetic coding sequence are synonymous codons selected from TABLE 1.

13. The method according to any one of claims 1 to 12, wherein at least 85% of codons in the first synthetic coding sequence and the second synthetic coding sequence are synonymous codons selected from TABLE 1.

14. The method according to any one of claims 1 to 13, wherein at least 90% of codons in the first synthetic coding sequence and the second synthetic coding sequence are synonymous codons selected from TABLE 1.

15. The method according to any one of claims 1 to 14, wherein at least 95% of codons in the first synthetic coding sequence and the second synthetic coding sequence are selected from TABLE 1.

16. The method according to any one of claims 1 to 15, wherein about 98% or more of the codons in the first synthetic coding sequence and the second synthetic coding sequence are synonymous codons selected from TABLE 1.

17. The method according to any one of claims 1 to 16, wherein the composition is formulated with a pharmaceutically acceptable carrier or excipient.

18. The method according to any one of claims 1 to 17, wherein the composition is administered with an adjuvant.

19. The method according to any one of claims 1 to 17, wherein the composition is administered without an adjuvant.

20. The method according to any one of claims 1 to 19, wherein the composition is formulated for intradermal administration.

21. The method of any one of claims 1 to 20, wherein the subject is a human.

22. The method according to any one of claims 1 to 21, wherein between about 30 μg and about 1000 μg of synthetic construct is administered per dose.

23. The method according to claim 22, wherein multiple doses are administered as part of a treatment regimen.

24. The method according to claim 23, wherein doses are administered daily, weekly, fortnightly, monthly, bimonthly or any time in between.

25. Use of a construct system for treating an HSV-2 infection in a subject, wherein the a construct system that comprises a first construct and a second construct, wherein the first construct comprises a first synthetic coding sequence that is distinguished from a wild-type HSV gD2 coding sequence by replacement of selected codons in the wild-type HSV gD2 coding sequence with synonymous codons that have a higher immune response preference than the selected codons, wherein codon replacements are selected from TABLE 1, wherein at least 70% of the codons of the first synthetic coding sequence are synonymous codons according to TABLE 1, and wherein the first synthetic coding sequence is operably connected to a regulatory nucleic acid sequence, and wherein the second construct comprises a second synthetic coding sequence that is distinguished from a wild-type HSV gD2 coding sequence by replacement of selected codons in the wild-type HSV gD2 coding sequence with synonymous codons that have a higher immune response preference than the selected codons, wherein codon replacements are selected from TABLE 1, wherein at least 70% of the codons of the first synthetic coding sequence are synonymous codons according to TABLE 1, and wherein the second synthetic coding sequence is operably connected to a regulatory nucleic acid sequence and to a nucleic acid sequence that encodes a protein-destabilizing element that increases processing and presentation of the polypeptide through the class I major histocompatibility (MHC) pathway.

26. The use of claim 25, wherein the construct system is prepared or manufactured as a medicament for this purpose.
